(19) United States
(12) Patent Application Publication
(10) Pub. No.: US 2021/0035662 A1
(43) Pub. Date: Feb. 4, 2021

(54) SYSTEMS AND METHODS FOR IMPROVING DISEASE DIAGNOSIS USING MEASURED ANALYTES

(71) Applicant: Otraces, Inc., Gaithersburg, MD (US)

(72) Inventors: Glaina Krasik, Montgomery Village, MD (US); Keith Lingenfelter, Potomac, MD (US)

(21) Appl. No.: 16/637,576

(22) PCT Filed: Aug. 9, 2018
(86) PCT No.: PCT/US2018/046056

§ 371 Date: Feb. 7, 2020

Related U.S. Application Data
(60) Provisional application No. 62/542,865, filed on Aug. 9, 2017.

(51) Int. Cl. G16H 10/40; G16H 50/20; G16H 50/30

(52) U.S. Cl. CPC G16H 10/40; G16H 50/30; G16H 50/20

(57) ABSTRACT

Systems and methods for diagnosing diseases such as prostate cancer, breast cancer, lung cancer, ovarian cancer, and their stages are disclosed. In certain embodiments, the disclosed systems and methods collect patient samples, calculate concentrations and Proximity Scores of biomarkers, and use those calculations to produce a training set model that is used to correlate biomarker concentrations and Proximity Scores to disease diagnoses and disease states (e.g. cancer stages). In certain embodiments, the correlation techniques used include simple regression, a ROC curve area maximization, a topology stabilization, or a Spatial Proximity Correlation analysis.

### CROSS-REFERENCE TO RELATED APPLICATIONS

This application claims the benefit of U.S. Provisional Application No. 62/542,865, filed Aug. 9, 2017, the entirety of which is hereby incorporated by reference herein.

A related patent application, International Application No. PCT/US2014/000041, filed Mar. 13, 2014, (hereby incorporated by reference in its entirety herein) describes methods for improving disease prediction using an independent variable for the correlation analysis that is not the concentration of the measured analytes directly but a calculated value termed “Proximity Score” that is computed from the concentration but is also normalized for certain age (or other physiological parameters) to remove age drift and non-linearities in how the concentration values drift or shift with the physiological parameter (e.g., age, menopausal status, etc.) as the disease state shifts from not-disease to disease.

### FIELD OF THE INVENTION

The present invention relates to methods for improving the accuracy of disease diagnosis and to associated diagnostic tests involving the correlation of measured analytes with binary outcomes (e.g., not disease or disease), as well as higher-order outcomes (e.g., one of several phases of a disease).

### BACKGROUND OF THE INVENTION

Correlation methods where three or more independent variables are used to correlate a binary outcome (such as the presence or absence of a given disease) commonly use the Spatial Proximity Correlation Method (also called cluster or neighborhood search method), the regression method and the wavelet methods. In the case of disease prediction, common constituents of blood or serum are measured and a correlation is attempted using these concentrations as independent variables for various disease state predictions. In the case of a given disease state where the outcome is either “disease” or “not disease,” the logistic regression method is commonly used. Other techniques involve, for example, genetic algorithms. The predictive power of these methods is highly dependent on the constituent analytes chosen for the method. Persons skilled in the art recognize that many analytes and parameters that would seem to have predictive power do not improve diagnostic and analytical power in practice.

The regression method uses trends in the independent variables to correlate with the outcomes. The linear method is based on linear trends, while logistic regression is based upon logarithmic trends. In biological disease prediction, most commonly, logistic regression is used to determine outcomes.

The group Spatial Proximity method surveys a variable correlation topology for grouping of like outcomes. The Spatial Proximity method has the advantage that it can find correlations where trends are not contiguous but have topology local reversals in trends. This method, though, is highly non-linear and susceptible to highly local variable outcomes with small measurement errors that can be more predictive in biological uses. Additionally, both methods discussed here can be combined with a Spatial Proximity method applied at a small scale to create a consolidated overall regression method.

However, some independent variables that would logically seem to have a correlation in practice do not show a predictive trend. Thus, what has been needed is an approach that improves diagnostic accuracy by utilizing patient-specific and population-specific variables that heretofore have not contributed useful information to the diagnosis of disease states.

Much research has been done to find biomarkers that alone or in combination can predict disease states with sufficient reproducibility and predictive power for clinical use. This research has had limited or no success. High Abundance Proteins (HAPs) have been heavily researched to find a single protein that can make this prediction. Numerous examples have been found but none have sufficiently low levels of false negatives to allow screening patients for the disease with the marker.

As a result, such single biomarkers are used for only therapy monitoring with the exception of PSA for prostate cancer. This test requires that the concentration that indicates a biopsy would be appropriate be heavily skewed to lower false negatives resulting in very high levels of false positives. As much as 80% of the men who are indicated to need biopsy are actually negative for prostate cancer. DNA markers also have been found to be very good in some cases for a sub-type of a cancer, but again are not suitable for screening for the same reasons as the HAPs noted above.

Using multiple proteins, proteomic approaches have also been investigated. This work has focused on, again, HAPs or on high level effecter proteins. This work has been dominated by multiplex methods of protein measurement such as immunoassays, chips and mass spectrophotometry. Very early work has found some success with ovarian cancer. However, a problem with all of these methods is that many of the proteins selected do not have a strong correlation with progression from healthy to disease (and many do not have a known biological connection with a disease state, for example, as typically is the case with mass spectrometry). Furthermore, mass spectrometry suffers a serious over-sampling problem due to the fact that the whole serum sample is interrogated by the spectrophotometer for protein levels and thus the training of the correlation algorithm is difficult. In the mass spectrometry case, the whole serum sample may have over 200 proteins and 10,000 mass spec peaks.

What also has been needed in the diagnostic field are techniques that utilize lower abundance proteins that are more useful for diagnostic purposes than are HAPS, as well as analytical techniques that provide for analysis of low abundance biomarkers.

Diagnostic Medicine has long searched for a simple, accurate serum based blood test to detect cancer and to detect whether the cancer is severe or quiescent. For example, the current test, the Prostate Specific Antigen (PSA) for prostate cancer suffers from very high false positive rates with a true detection false negative rate of as high as 1 in ten men. This test has a predictive power of about 57%. Furthermore, men who are diagnosed with low grade prostate cancer may not need treatment for many years or for the rest of their lives. This diagnosis can today only be accurately obtained through PCa biopsy. The current PSA test indiscriminately sends all (90%, one in ten are missed) with a PSA level above 4.0 ng/ml to biopsy and only about 20% have any PCa regardless of Gleason Score. Beyond this, men with low grade PCa are at risk of converting to high grade in later years of life and the only sure way of diagnosing this accurately is with more biopsies. Additional biopsies for monitoring are not acceptable to the medical community due to cost and are unacceptable to the patient due to pain and side effects. Ongoing monitoring of men with low grade PCa is thus done with periodic PSA test accompanied by digital rectal exams (DRE) and sometimes CT scans. In many cases, prophylactic treatment is done, removal of the prostate when it may not be medically necessary. This patent teaches a new serum based test that can distinguish men without PCa from high grade and can detect men with low grade PCa who convert in later life. Additionally, it teaches a blood based test that can discriminate early stage solid tumor cancers such as lung or breast or cancer stage.

The current PSA screening test was approved in the mid 1980′s and is now off patent. The new so called 4K Score test offered as a “Lab developed Test” by OPKO does not have regulatory approval. It purports to detect men with high grade PCa, separating this condition from low grade PCa. Generally, high Grade PCa is considered to be Gleason Scores (obtained at biopsy) of 7(4+3) or higher (8, 9 or 10), whereas low grade is considered to be 7 (3+4) or lower. The PSA test for detecting men with all grades of PCa is about 57% predictive power, or for a sensitivity of 90% the false positive rate is about 80% (1 out of 4 positives are actually negative). The 4K Score test has a predictive power of about 64%. Thus, for a 1 out 10 false negative rate, the false positive rate is about 50% or about 5 out of 10 are actually negative. This is the current state of PCa diagnostic testing in medicine today.

Currently there are no regulatory approved methods for detecting diseases such as lung cancer and breast cancer by simple blood test. Furthermore, these diseases can only assessed for severity by biopsy. We also propose additional tests for assessing tumor stage using again active cytokines in the tumor microenvironment using blood serum as a proxy for these proteins.

In order to alleviate these and other deficiencies in the art, a new test is described herein exemplarily using active cytokines in the tumor microenvironment, where blood serum acts as a proxy for these proteins.

### BRIEF DESCRIPTION OF THE DRAWINGS

A more complete appreciation of the invention and many of the attendant advantages thereof will be readily obtained as the same becomes better understood by the reference to the following detailed description when considered in connection with the accompanying figures, wherein:

FIG. 1 is a chart that displays the surge in biomarker concentrations by Gleason Score for prostate cancer;

FIG. 2 is a chart that displays the surge in biomarker concentrations by Gleason Score for lung cancer;

FIG. 3 is a chart that displays the average up-regulation of biomarker concentrations corresponding to the stages of breast cancer;

FIG. 4 is a chart that displays the VEGF Receiver Operator Characteristic (“ROC”) curve for aggressive prostate cancer vs. Not-Cancer;

FIG. 5 is chart that displays the TNFα ROC curve for aggressive prostate cancer vs. Not-Cancer;

FIG. 6 is a chart that displays the PSA ROC curve for aggressive prostate cancer vs. Not-Cancer;

FIG. 7 is a chart that displays the IL 6 ROC curve for aggressive prostate cancer vs. Not-Cancer;

FIG. 8 is a chart that displays the IL 10 ROC curve for late-stage lung cancer vs. early-stage lung cancer;

FIG. 9 is a chart that displays the IL 6 ROC curve for late-stage lung cancer vs. early-stage lung cancer;

FIG. 10 is a chart that displays the VEGF ROC curve for late-stage lung cancer vs. early-stage lung cancer;

FIG. 11 is a chart showing the results of the blind tests with two samples that failed the topology instability test and were corrected with the incongruent algorithm in accordance with an embodiment of the disclosed diagnostic method;

FIG. 12 is a chart showing the results of the clinical study for breast cancer in this case the training set cancer scores are shown for Training Set Model I using 10 bi-marker planes in accordance with an embodiment of the disclosed diagnostic method;

FIG. 13 is a chart showing the results of the clinical study for breast cancer in this case the training set cancer scores are shown for Training Set Model II using 105 bi-marker planes in accordance with an embodiment of the disclosed diagnostic method;

FIG. 14 is a chart showing the results with actual diagnosis for the blind samples run the clinical study in accordance with an embodiment of the disclosed diagnostic method;

FIG. 15 is a chart showing a bi-marker plane for one of the ten such planes showing Proximity Scores of two of the biomarkers used in accordance with an embodiment of the disclosed diagnostic method;

FIG. 16 is a chart showing a bi-marker plane with training set data points in accordance with an embodiment of the disclosed diagnostic method;

FIG. 17 is a chart showing a bi-marker plane without the training set data points in accordance with an embodiment of the disclosed diagnostic method;

FIG. 18 is a chart showing a bi-marker plane with shaded area where influence is lowered for immune system response in accordance with an embodiment of the disclosed diagnostic method;

FIG. 19 is a chart showing a bi-marker plane with shaded area where influence is lowered for topology stability problems in accordance with an embodiment of the disclosed diagnostic method;

FIG. 20 is a chart showing a bi-marker plane with shaded area where influence is lowered for known assay measurement uncertainty in accordance with an embodiment of the disclosed diagnostic method;

FIG. 21 is a chart showing the results of the blind tests with two samples that failed the topology instability test and were corrected with the incongruent algorithm in accordance with an embodiment of the disclosed diagnostic method;

FIG. 22 is a flow chart showing the general logical pathway followed by the software of the present invention, in accordance with an exemplary embodiment;

FIG. 23 is a flow chart that represents the process of constructing the Training Set Model (or diagnostic model) and then producing diagnostic scores for blind samples that assess risk of having the disease state or non-diseased state;

FIG. 24 shows a typical population distribution, in this case for the cytokine, Interleukin 6 (IL 6);

FIG. 25 is a chart showing a transformation of biomarker concentration to a Proximity Score (one type of pseudo-concentration); and

FIG. 26 shows a representative diagram of the hardware used in implementing the software of the invention, in accordance with an exemplary embodiment.

### SUMMARY OF THE INVENTION

Without limiting the foregoing, in a preferred embodiment, the invention relates to improving the predictive power and diagnostic accuracy of methods for predicting disease states using multi-variable (multi-variant) correlation methods. These methods include proteomic, metabolomic and other techniques that involve the determination of levels of various biomarkers as found in bodily fluids and tissue samples.

Various embodiments contemplated by the inventors and discussed in this application include the use of meta-variables, particularly using methods that adjust the influence of measured biomarker analytes on a correlation score. Such meta-variables may be identified based upon special knowledge of immune system response and knowledge of possible measurement errors. These methods can be applied to either the construction of the training set model or to the blind samples under diagnosis.

In one aspect, the present invention relates to a method for diagnosing a disease, comprising the steps of: a) determining the concentrations of at least three predetermined analytes in a blind sample from a subject; b) selecting one or more meta-variable associated with the subject, which varies in a population associated with the subject for members of the population who are known either to have or not have the disease; c) transforming the concentrations of the analytes as a function of one or more population distribution characteristics and the one or more meta-variables to compute a Proximity Score that represents each analyte; d) comparing the Proximity Scores to a training set model of Proximity Scores determined for members of the population who are known either to have or not have the disease; and e) determining whether the comparison indicates that the subject has the disease. It is contemplated that the step (a) of determining the concentrations (or levels) of predetermined analytes may be performed in a separate time and place from the remaining steps of the method. Similarly, other step(s) of the method may be practiced in whole or in part at separate times and places. Accordingly, the present inventors contemplate as their invention a method that contains only steps (b)-(f).

### DETAILED DESCRIPTION OF THE PREFERRED EMBODIMENTS

In describing preferred embodiments of the invention illustrated in the drawings, specific terminology will be resorted to for the sake of clarity. However, the invention is not intended to be limited to the specific terms so selected, and it is to be understood that each specific term includes all technical equivalents that operate in a similar manner to accomplish a similar purpose. Several preferred embodiments of the invention are described for illustrative purposes, it being understood that the invention may be embodied in other forms not specifically shown in the figures.

For the purposes of this application, specific terminology is used to better describe the preferred embodiments of the invention, which is defined below:

“Analytical Sensitivity” is defined as three standard deviations above the zero calibrator. Diagnostic representations are not considered accurate for concentrations below this level. Thus, clinically relevant concentrations below this level are not considered accurate and are not used for diagnostic purposes in the clinical lab.

“Baseline Analyte Measurement for an Individual” is a measurement set of the biomarkers of interest for the transition of an individual patient from the not disease state to the disease state, measured for a single individual multiple times over a period of time. The Baseline Analyte Measurement for the not disease state is measured when the individual patient does not have the disease, and alternatively, the Baseline Analyte Measurement for the disease state is determined when the individual patient has the disease. These baseline measurements are considered unique for the individual patient and may be helpful in diagnosing the transition from not disease to disease for that individual patient. The Baseline Analyte Measurement for the disease state may be useful for diagnosing the disease for the second or higher occurrence of the disease in that individual.

“Biological Sample” means tissue or bodily fluid, such as blood or plasma, that is drawn from a subject and from which the concentrations or levels of diagnostically informative analytes (also referred to as markers or biomarkers) may be determined.

“Biomarker” or “Marker” means a biological constituent of a subject's biological sample, which is typically a protein or metabolomic analyte measured in a bodily fluid such as a blood serum protein. Examples include cytokines, tumor markers, and the like. The present invention also contemplates other indicia as “biomarkers” and “markers,” including but not limited to: height, eye color, geographic factor, environmental factors, etc. In general, such indicia will include any measurements or attributes that vary within a population and remain measurable, determinable, or observable.

“Blind Sample” is a biological sample drawn from a subject without a known diagnosis of a given disease, and for whom a prediction about the presence or absence of that disease is desired.

“Disease Related Functionality” is a characteristic of a biomarker that is either an action of the disease to continue or grow or is an action of the body to stop the disease from progressing. In the case of cancer, a tumor will act on the body by requesting blood circulation growth to survive and prosper, and the immune system will increase pro-inflammatory actions to kill the tumor. These biomarkers are in contrast to tumor markers that do not have Disease Related Functionality, but are sloughed off into the circulatory system and thus can be measured. Examples of Functional Biomarkers would be Interleukin 6 which turns up the actions of the immune system, or VEGF which the tumor secretes to cause local blood vessel growth. Whereas a non-functional example would be CA 125. That is a structural protein located in the eye and human female reproductive tract and has no action by the body to kill the tumor or action by the tumor to help the tumor grow.

“Limit of Detection” (LOD) is defined as a concentration value 2 standard deviations above the value of the “zero” concentration calibrator. Usually the zero calibrator is run in 20 or more replicates to get an accurate representation of the standard deviation of the measurement. Concentration determinations below this level are considered as zero or not present for example, for a viral or bacterial detection. For purposes of the present invention, 1.5 standard deviations can be used when samples are run in duplicate, although the use of 20 replicates is preferred. Diagnostic representations requiring a single concentration number are generally not rendered below this level. Measurements at the level of Limit of Detection statistically are at a 95% confidence level. Predictions of disease state using the methods discussed here are not based upon a single concentration and predictions are shown to be possible at measurements levels below the concentration based LOD.

“Low Abundance Proteins” are proteins in serum at very low levels. The definition of this level is not clearly defined in the literature but as used in this specification, the level would be less than about 1 picogram/milliliter in blood serum or plasma and other body fluids from which samples are drawn.

“Meta-variable” means information that is characteristic of a given subject, other than the concentrations or levels of analytes and biomarkers, but which is not necessarily individualized or unique to that subject. Examples of such meta-variables include, but are not limited to, a subject's age, menopausal status (pre-, peri- and post-) and other conditions and characteristics such as pubescence, body mass, geographic location or region of the patient's residence, geographic source of the biological sample, body fat percent, age, race or racial mix, or era of time.

“Population Distribution” means the range of concentrations of a particular analyte in the biological samples of a given population of subjects. A specific “population” means, but is not limited to: individuals selected from a geographic region, a particular race, or a particular gender. And the population distribution characteristic selected for use as described in this application further contemplates the use of two distinct subpopulations within that larger defined population, which are members of the population who have been diagnosed as having a given disease state (disease subpopulation) and not having the disease state (non-disease subpopulation). The population can be whatever group in which a disease prediction is desired. Moreover, it is contemplated that appropriate populations include those subjects having a disease that has advanced to a particular clinical stage relative to other stages of disease progression.

“Population Distribution Characteristics” are determinable within the population distribution of a biomarker, such as the mean value of concentration of a particular analyte, or its median concentration value, or the dynamic range of concentration, or how the population distribution falls into groups that are recognizable as distinct peaks as the degree of up or down regulation of various biomarkers and meta-variables of interest are affected by the onset and progression of a disease as a patient experiences a biological transition or progression from the non-disease to disease state.

“Predictive Power” means the average of sensitivity and specificity for a diagnostic assay or test, or one minus the total number of erroneous predictions (both false negative and false positive) divided by the total number of samples.

“Proximity Score” means a substitute or replacement value for the concentration of a measured biomarker and is, in effect, a new independent variable that can be used in a diagnostic correlation analysis. The Proximity Score is related to and computed from the concentration of measured biomarker analytes, where such analytes have a predictive power for a given disease state. The Proximity Score is computed using a meta-variable adjusted population distribution characteristic of interest to transform the actual measured concentration of the predictive biomarker for a given patient for whom a diagnosis is desired, as disclosed in International Publication No. WO 2017/127822 and International Publication No. WO 2014/158287. “Proximity Score” and “pseudo-concentration” have the same definition and may be used interchangeably.

“Specificity” is a true false positive rate of a test. It is mathematically one minus the false positive number of measurements of the test divided by the total number of true negative samples measured.

“Incongruent Training Set Model” (or “Secondary Algorithm”) is a secondary training set model that uses a different phenomenological data reduction method such that individual points on the grids of the bi-marker planes are not likely to be unstable in both the primary correlation training set model and this secondary algorithm.

“Spatial Proximity Correlation Method” (or Neighborhood Search or Cluster Analysis) is a method for determining a correlation relationship between independent variables and a binary outcome where the independent variables are plotted on orthogonal axes. The prediction for blind samples is based upon proximity to a number (3, 4, 5 or more) of so called “Training Set” data points where the outcome is known. The binary outcome scoring is based upon the total distance computed from the blind point on the multi-dimensional to Training Set points of opposite outcome. The shortest distance determines the scoring of the individual blind data point.

This same analysis can be done on bi-marker planes cut through the multidimensional grid where the individual bi-marker plane score is combined with the score of the other planes to yield a total. This use of cuts or two dimensional orthogonal projections through the space can reduce computation time.

“Training Set” is a group of patients (200 or more, typically, to achieve statistical significance) with known biomarker concentrations, known meta-variable values and known diagnosis. The training set is used to determine the axes values “Proximity Scores” of the “bi-marker” planes as well as score grid points from the Spatial Proximity analysis that will be used to score individual blind samples.

“Training Set Model” is an algorithm or group of algorithms constructed from the training set that allows assessment of blind samples regarding the predictive outcome as to the probability that a subject (or patient) has a disease or does not have the disease. The “training set model” is then used to compute the scores for blind samples for clinical and diagnostic purposes. For this purpose, a score is provided over an arbitrary range that indicates percent likelihood of disease or not-disease or some other predetermined indicator readout preferred by a healthcare provider who is developing a diagnosis for a patient.

“Receiver Operator Characteristic (ROC) curve” is a graphical method for representing the performance of a signaling method used for decision making where there is a tradeoff between the false positive, false negative rates and the intensity of the detecting signal. In this graphical representation, the ordinate of the plot contains the sensitivity of the test method, and the abscissa has the false positive rate. For biomarkers (or signals) with upward action to the disease trip point, the curve will be above a 45° null line originating at the origin (0,0) of the plot to the upper right of the plot (1.0,1.0). The area under the curve indicates how good the biomarker is at making the prediction.

“ROC Curve ‘Area Under the Curve’ (AUC)” is the area under the biomarker characteristic curve and the abscissa. For a perfectly useless biomarker, the AUC will be 0.5 and its the area under the 45° null line referred to above. A perfect test has an AUC of 1.0 and extends from the origin up the ordinate to the 100% sensitivity point and then across the ROC curve to the 1.0, 1.0 point at the upper right.

“Tumor Microenvironment” is bathed in the tumor interstitial fluid (TIF), is the cellular environment in which the tumor exists, including surrounding blood vessels, immune cells, fibroblasts, bone marrow-derived inflammatory cells, Lymphocytes, signaling molecules and the extracellular matrix.

“Tumor Marker” is a protein marker that is sloughed off into the TME or blood supply that has no apparent function, is either the tumor's growth by tumor secretions or the tumor's suppression by the immune system.

In recent years, cancer immunotherapy research has become increasingly interested in the Tumor Microenvironment (TME) which provides an ideal R&D platform for the development and evolution of new therapies and represents a potentially vast storehouse of diagnostic content. The TME, which is bathed in the tumor interstitial fluid (TIF), is the cellular environment in which the tumor exists, including surrounding blood vessels, immune cells, fibroblasts, bone marrow-derived inflammatory cells, Lymphocytes, signaling molecules and the extracellular matrix.

The TIF is also the transport fluid linking the tumor (and the TME) to the blood supply, and is important as it is the “battlefield messenger” for the active proteins that the immune system uses to try to suppress the tumor or the tumor expresses to assist its growth. These competing proteins, or cytokines, which are constantly at war with one another, fall into several functional categories of low level signaling proteins: pro- and anti-inflammatory, anti-tumor genesis (or cell apoptosis), angiogenesis and vascularization.

Although recognized as a potential source of rich diagnostic information, development of TIF analysis as a cancer screening modality has not progressed as sampling this fluid is very difficult and in order to do so means that the location of the tumor is known and therefore whether a tumor already exists. More challenging is detecting the presence of the TME/TIF and thus a malignancy without this knowledge. This requires a more accessible fluid for clinical diagnosis, such as blood serum, coupled with analysis of multiple proteins, known as proteomics, which may presumably be correlated to the presence or absence of disease. Serum presents some problem in this regard, as it is more an amalgam of the conditions in the patient's body than a direct pathway to detect the presence of an active TME (and thus a tumor).

In this disclosure, we discuss a method for analyzing specific cytokines present in serum as an accurate proxy for the proteins active in the TME and TIF. The method involves several steps including two proprietary processes, termed proteomic noise suppression and multidimensional (or spatial) correlation. The method we describe can yield an accurate proxy for the actions of the proteins found in the TIF and thus is useful for detecting the presence of an active TME within the organism and thus a tumor. In essence, this method isolates the signature of the TME in the serum and indicates the presence (or not) of an active TME, indicating that an active tumor is present. Beyond this, the method measures the modulation of these proteins, which yields valuable information about the status of the tumor, degree of aggressive action and stage, as well as information about the immune system's progress in suppressing the tumor.

Biomarkers of Interest

The biomarkers of interest in this disclosure are pro-inflammatory (Interleukin 6, IL 6, or others); anti-inflammatory (Interleukin 10, IL 10, or others) Antitumor or tumor killing cytokines (tumor necrosis factor alpha, TNFα, or others), and circulatory growth factors such as angiogenesis (interleukin 8, IL 8, or others) and vascularization cytokines (vascular endothelial growth factor, VEGF or others. These are cytokines with directly related functionality of the immune system's response to the tumor or the tumor's action on the body. Vascularization factors, VEGF, is the tumor's action to grow the circulatory system within the bulk of the growing tumor. Tumor anti-genesis factors, TNFα, is the action of the immune system to kill the tumor (apoptosis) and pro-inflammatory factor, IL 6, is the mediator of overall immune system's action. Anti-inflammatory, IL 10, is secreted by the Tumor into the Tumor interstitial fluid to suppress the immune system. And finally, angiogenesis factors like IL 8 are secreted by the tumor to grow vascularization in the surrounding tissue.

In general, cancer is a pro-inflammatory disease in which factors such as IL-6 are upregulated. However, in the three cases described herein the tumor in its later stage secretes an anti-inflammatory cytokine into the tumor interstitial fluid (and thus the blood). This action is shown to occur in the later stages of cancer, Stage 3 or 4 in lung and breast, and at higher Gleason Score prostate cancer (Gleason 8, 9 or 10). At this point the anti-inflammatory action tends to down regulate the pro-inflammatory response of the organism's immune system. In some cases, with Breast cancer, the angiogenesis response is also suppressed in later stages. Finally the vascularization action of the tumor increases as one might expect as the tumor grows in size, breast and lung stage 3 or 4 and for prostate cancer Gleason Scores 8, 9 and 10. All of these actions occurring within the tumor microenvironment can be discerned by sampling the serum of the organism and applying the methods described in referenced International Publication Nos.: WO 2017/127822 and WO 2014/158287.

In the particular case of cancer, a high degree of recent therapeutic research interest has focused on the so-called “tumor microenvironment” (TME) for development of treatment modalities. Suppression or enhancement of the regulation of proteins active in the tumor interstitial fluid (TIF) found within the TME is thought to be a fruitful development path for these treatments. Proteins in the TIF that have been found to be good indicators are generally from five functional cytokine groups: pro- or anti-inflammatory, anti-tumor genesis (or cell apoptosis), angiogenesis and vascularization.

Measurement of the activity of these proteins can provide insight into tumor activity and therapeutic impact. For example, treatment modalities that promote or suppress the protein activity can be monitored in the TIF to determine efficacy. While appropriate for therapeutic applications, where the cancer is known to exist, sampling the TIF for diagnostic purposes has not been pursued. As the presence of TIF (and that of a TME) means, by definition, that the patient has an active tumor with a known location, its use as a diagnostic tool is moot. Beyond this, accessing these proteins for diagnosis when present in other bodily fluids, such as serum or urine, has not been considered because up until now the proteomic noise problem has rendered them unusable.

Herein we describe systems and methods for producing an accurate proxy for the TME activity that uses active proteins in the TIF, using an easily accessible proxy fluid—in this case serum (other fluids, such as urine are possible). It should be noted that serum is an amalgam of the conditions of the overall organism (termed “proteomic noise”) and is not specific to the tumor. The methodology we propose also eliminates proteomic noise, allowing an accurate assessment of the patient's condition.

In general, the systems and methods disclosed herein involve: 1) selecting active TIF proteins that are indicative of conditions in the TME, 2) measuring these proteins in the serum proxy, 3) suppressing the proteomic noise to cleanly identify cancer-related activity in the proteins, 4) then performing a correlation method that amplifies the actions of these proteins in a multidimensional matrix, and 5) scoring the protein activity to indicate the presence or absence of cancer, and if present, its development stage. This is done first to create a training set, representative of the population as a whole, that serves as a yardstick against which individual samples are then compared to determine their status—either diseased or disease free.

Biomarker Combined Actions

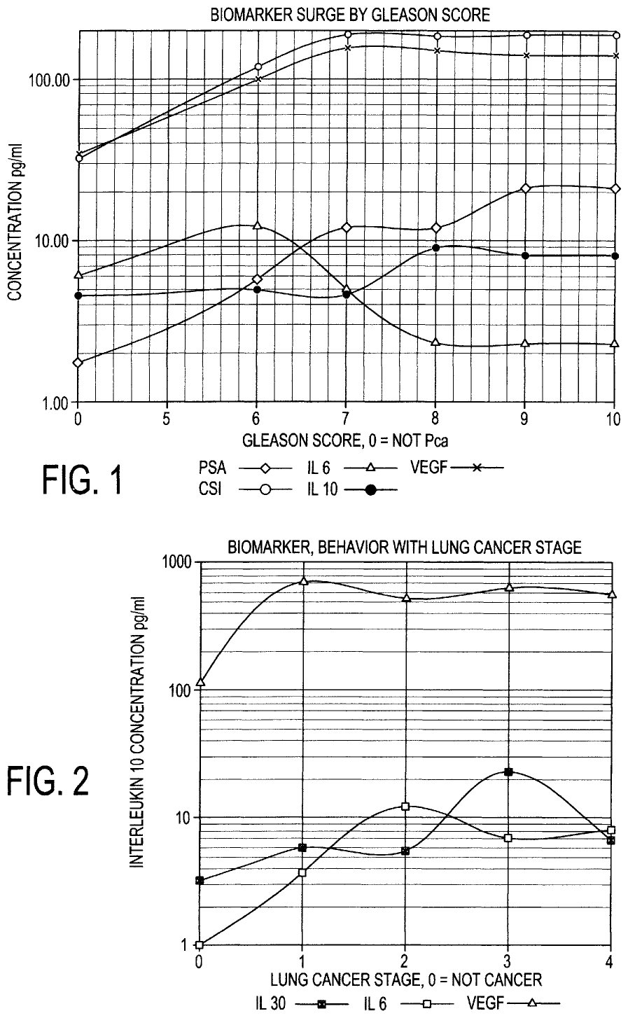

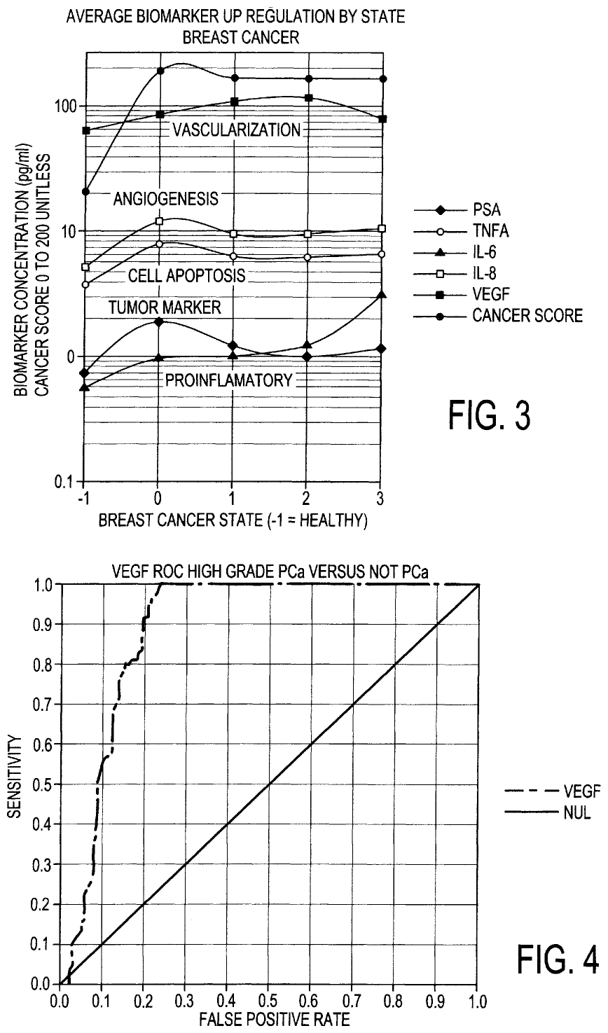

These cytokine biomarkers are very active in the high grade prostate cancer and compared to levels in “healthy” men are highly up or down regulated and thus very good indications of disease status. Also note that they are active in Lung and Breast Cancer. FIGS. 1, 2 and 3 show this action as the tumor progresses. Note that in non-small cell lung and prostate cancer, shown in FIGS. 1 and 2, IL 6 down-regulates in late stage cancer or at high Gleason Score 8, 9 or 10. Also note that in both cases, in the transition from low-grade lung or low Gleason Score prostate cancer, the increased Interleukin 10 secreted by the tumor results in the down regulation of IL 6. Also note that secretion of IL 10 into the tumor interstitial fluid and thus the blood is associated with poor patient prognosis. This usually means later stage breast cancer is present. The combination of IL 6 and IL 10 in a correlation analysis of the disease state is thus improved by using the combination of a pro-inflammatory and anti-inflammatory cytokines. Furthermore, note that vascularization cytokines continue to up regulate in general as the tumor becomes later stage or more aggressive.

Three of these biomarkers have unique ROC curve characteristics that are not common to tumor biomarkers. They have a flat portion at 100% sensitivity for certain lower levels of the biomarker's concentrations. They also have fairly large areas under the curve (AUC), indicating they are very good biomarkers for this disease, high grade prostate cancer (PCa) versus not PCa. One of them has a straight vertical section going up the ordinate from [0, 0], indicating that samples is this signal range must have PCa, zero false positive rate.

In the scientific literature, there are limited publications for several of the biomarkers of choice referred to in this specification. And there is nothing related to the detection of high grade PCa versus the general population, that is, patients without PCa. In the case of VEGF, the literature does indicate an up-regulation of the biomarkers but nothing is stated as to the stage of the cancer or especially Gleason Score. Much of the literature is confined to VEGF's use as a prognosticator for treatment for men already diagnosed with PCa. TNFα also has no reference to differentiation of biomarker actions related to stage or especially Gleason Score of the tumor. A scientific survey for Interleukin 6 yields the same information. In fact, at low grade PCa, some literature indicates a slight up regulation of the biomarker. Our measures do not bear this out, as a slight down regulation is seen at low grade PCa, but a very strong down regulation is seen at high Gleason Score PCa, making this cytokine, along with the others, a strong indicator of the presence of high grade PCa. Much of the literature involves using these biomarkers as prognosticators in men with PCa and in vitro expression of the protein from PCa cell lines and looking into methods for suppression of the expression (esp. VEGF) for treatment purposes.

ROC Curves for Prostate Cancer

VEGF

The ROC curve for VEGF in Aggressive (Gleason Score 7 (4+3), 8, 9 and 10 is shown in FIG. 4. Notice the large flat portion of the ROC across the top where the sensitivity is 100%. Concentration levels for VEGF at this level of sensitivity or below about 50 pg/ml do not have high grade PCa, and none were detected. The AUC for this biomarker for this disease/not disease comparison is 0.87. In addition, the unique shape with no false positives below the 50 pg/ml level makes it a very good candidate for a high grade “PCa” versus “Not PCa” biomarker, as concentration levels below about 50 pg/ml show no PCa at all

TNFα

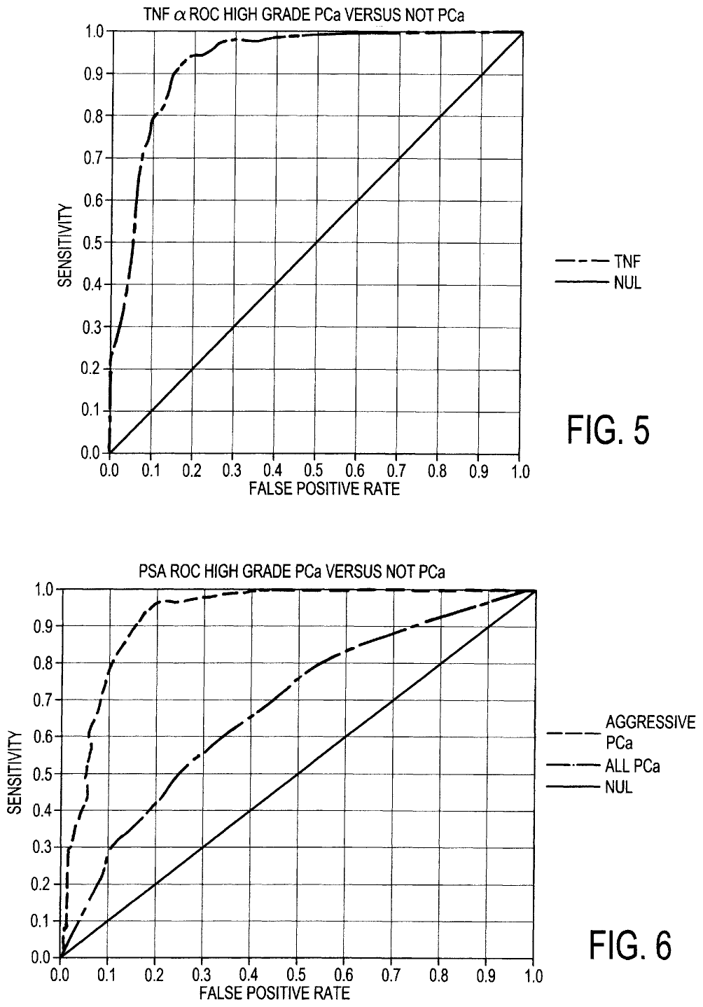

The comments on TNFα are the same regarding the character of the ROC curve for Aggressive (Gleason Score 7 (4+3), 8, 9 and 10, as shown in FIG. 5. In this case, the AUC is 0.85, again high and the same trip point is for no false negative results below about 6.5 pg/ml. TNFα also shows a portion of the curve that is at zero false positive rate (abscissa) for samples above about 9.85 pg/ml. In this region, there are no false positive results.

PSA

The comments on PSA are the same regarding the character of the ROC curve, for Aggressive (Gleason Score 7 (4+3), 8, 9 and 10, as shown in FIG. 6. In this case, the AUC is 0.85, again high and the same trip point is for no false negative results below about 2 ng/ml. The ROC curve for the common PSA assay for PCa all Gleason scores is shown for reference in green (shown as “All PCa”).

IL 6

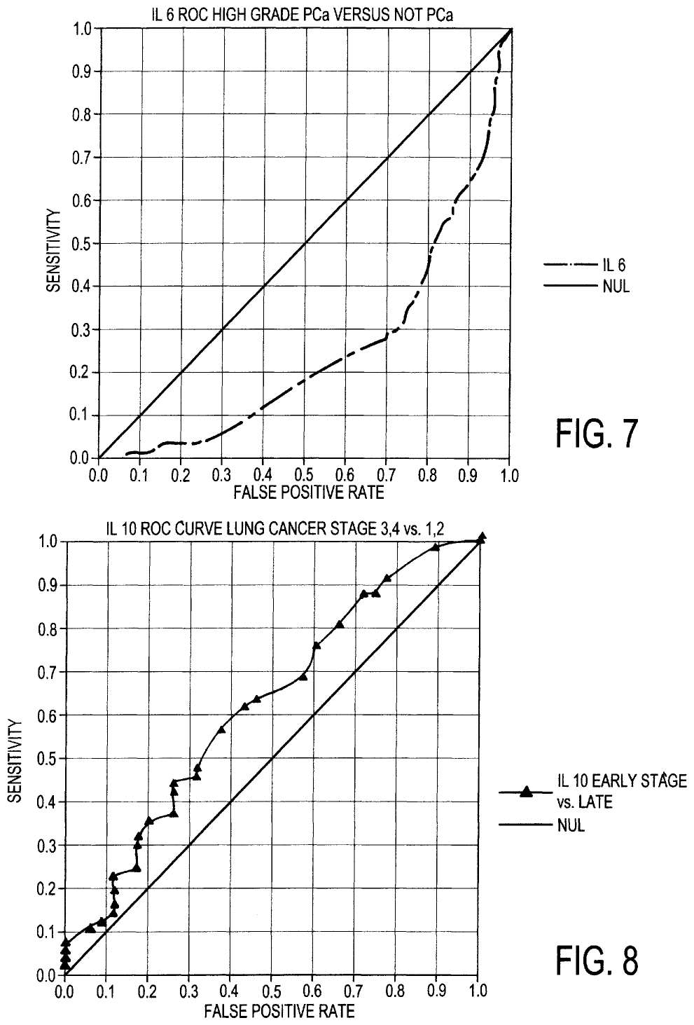

In contrast, IL 6 shows strong down-regulation in Aggressive (Gleason Score 7 (4+3), 8, 9 and 10, with an AUC about twice that of current PSA for detection of PCa, as shown in FIG. 7, in the general population (the curve must be inverted to account for this down regulation). Speculation would be that possibly the high grade PCa is effective at suppressing the immune system but that is not the point of this discussion. The fact is this biomarker shows strong down regulation. Limited literature indicates a slight up regulation with general PCa. Our measurement for all Gleason score PCa shows a slight down regulation. Nevertheless, at high Gleason score, the cytokine shows strong down regulation. The overall population of PCa is about 80% low grade, so when sampling PCa, the low grade will dominate the characteristics of the cohort. This down-regulation is likely caused by secretion of an anti-inflammatory cytokine (IL 10) when the tumor progresses to the aggressive Gleason Score 7 (4+3) and higher.

ROC Curves for Lung Cancer

IL 10

FIG. 8 shows the ROC curve for Interleukin 10 in the case of separating low grade (stage 1 and 2 from later stage 3, and 4 non-small cell lung cancer. Note it up regulates in the transition from early stage (1 and 2) to later stages (3 and 4). This corresponds to the down regulation of Interleukin 6 and is caused by the anti-inflammatory action of the tumor secreting IL 10 into the tumor microenvironment and subsequently into the blood stream.

IL 6

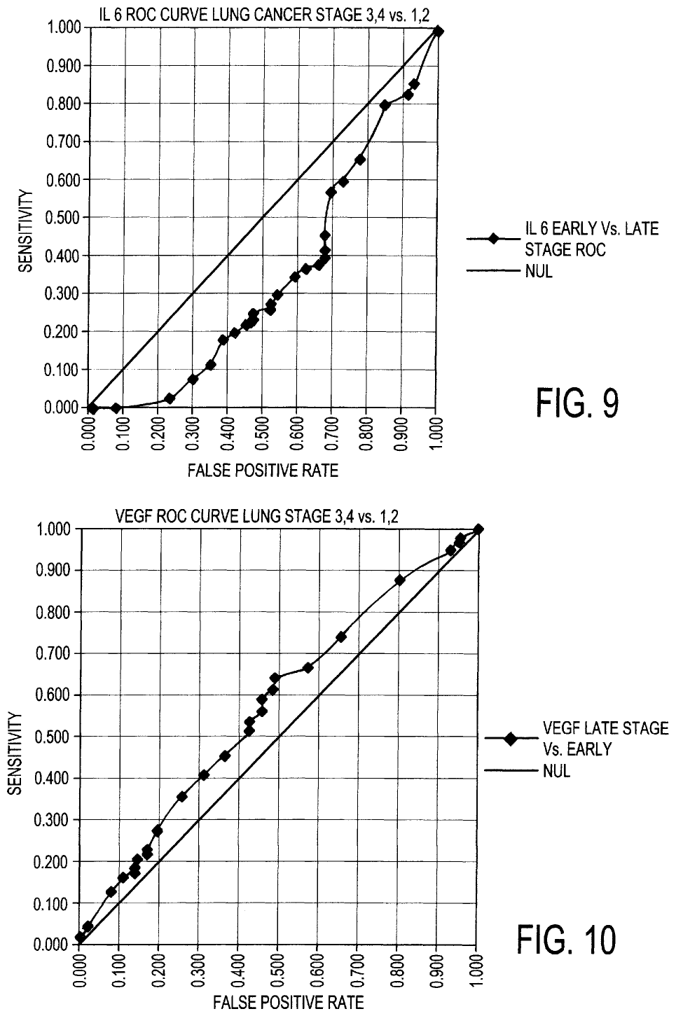

The ROC curve for IL 6 is shown in FIG. 9, again for the case of early stage (1 and 2) versus late stage (3 and 4) non-small cell lung cancer. As FIG. 9 demonstrates, this action of IL 6 is being suppressed by the anti-inflammatory action of the tumor.

VEGF

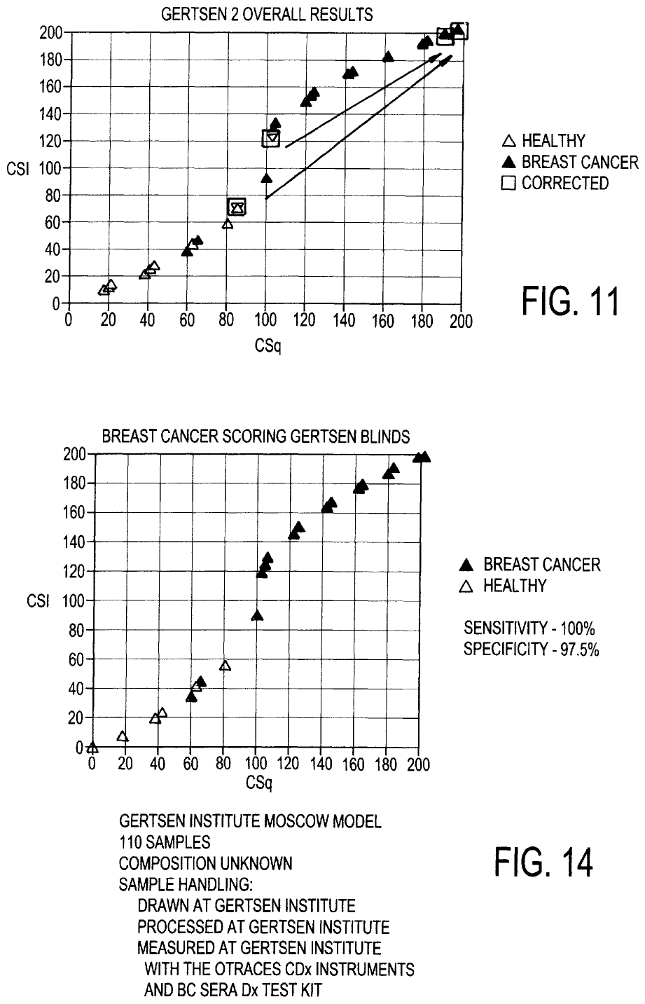

The ROC curve for VEGF is shown in FIG. 10, which demonstrates the up-regulation of the vascularization factor as found in other cancers as the tumor grows and progresses to later stages.

Test for Aggressive or Late-Stage vs. Non-Aggressive or Early-Stage Cancer

These biomarkers can be put together to develop a very simple proteomic algorithm for monitoring men with low grade Gleason Score 5, 6, or 7(3+4) prostate cancer for the transition to high grade, Gleason Score 7 (4+3), 8, 9 or 10 high grade PCa. Also, these biomarkers can discern early stage cancer, stage 1 or 2 from stage 3 or 4. The combination of IL 6 and IL 10 with opposing actions can produce (with a simple correlation method such as logistic regression) 80% predictive power. The addition of proteomic noise suppression and the Spatial Proximity Correlation Method will produce predictive powers of 90%. The addition of the action of VEGF to the biomarker panel will improve predictive power to 95% plus.

Test for Aggressive Prostate Cancer vs. Men Without Cancer

Indeed, VEGF by itself will produce a test with 76% predictive power, 100% sensitivity and 76% specificity (24% false positive rate). This simple model will simply exclude not PCa in those concentration ranges where the ROC curve excludes it and will include PCa in those zones again where the ROC curve includes it. Then, it will use a simple trip point count and count of positive and negative scoring of each biomarker not within the exclusion or inclusion criterion. The count must exceed 3 of 4 for those not pre-excluded or included. This simple model yields 100% of a representative sample's set of 100 PCa with High Gleason Score (defined as 7(4+3) and up) and 100 not PCa samples. Combining VEGF, IL 6, TNFα and PSA will produce predictive power of 90%. In addition this test will predict “not cancer” for men with elevated PSA but not the cytokines. These men have benign prostate hyperplasia or another non-malignant prostate condition and constitute the bulk of the numerous false positive results for the current PSA screening test for prostate cancer. A test incorporating these cytokines solves this false positive problem.

Predicting Cancer Stage

Data obtained from a breast cancer study supervised by the Gertsen Institute, Moscow were able to predict with high accuracy the stage of the breast cancer, using equipment and reagents described below. 189 samples with breast were obtained with stage information (0 through 4). The measurements were of the tumor marker PSA and the four cytokines noted, pro-inflammatory (IL 6), anti-tumor genesis (TNFα), angiogenesis (IL 8) and vascularization (VEGF). In this case, the goal was to score each sample for the likely staging information obtained from biopsy. The correlation methods are all binary in nature and cannot without some manipulation score four different outcomes. The stage groups were thus coupled into binary groups representing all stage groups; 1 plus 2, 3, 4; 2 plus 1, 3, 4; 3 plus 1, 2, 4 and 4 plus 1, 2, 3. All four groups were modeled and scored, using the age normalization, noise suppression and Spatial Proximity Correlation Methods described in the International Publication No. WO 2017/127822 and International Publication No. WO 2014/158287. The score for each individual sample was then computed using each individual group score of each sample added together with a weighting based upon each one's contribution to that group (1 or 1/3). This model produced 99% accuracy.

Several methods for improving the predictive power of traditional proteomics correlation methods for diagnosing disease have been described in this specification. These include: 1) using a meta-variable and Proximity Scores values for the correlation, and 2) using special knowledge of topology stability and assay measurement characteristics to adjust bi-marker plane influence in the training set model. Also, methods for finding and correcting blind sample stability problems unique to the particular training set model using an incongruent training set model are described. Additionally, methods for finding and correcting non-disease conditions that partially mimic the training set model for a given disease state are described. All of these methods are complimentary and can be used in concert. For example, adjusting the training set model for areas of high likelihood of instability cannot completely remove this problem from blind sample predictive calculations and thus both methods can be used for improvements in predictive power. The inventors have found that combining these methods can yield predictive powers above 95%, and the breast cancer study discussed in Example 1 below yielded over 98% predictive power (100% sensitivity, 97.5% specificity).

### EXAMPLE 1

### Clinical Study Assessing Breast Cancer Blood Test

The performance of the OTraces BC Sera Dx test kit and OTraces CDx Immunochemistry Instrument System (www.otraces.com) was evaluated in an experiment to assess the risk of the presence of breast cancer. The test kit measures the concentrations of five very low-level cytokines and tissue markers, and uses a training set model that was developed as described above to calculate scores, CS1 and CSq, for assessing the risk of breast cancer. The proteins measured were IL-6, IL-8, VEGF, TNFα and PSA. The experiment consisted of measuring about 300 patient samples split roughly 50% between breast cancer cases diagnosed by biopsy and 50% from patients putatively considered non-diseased (or in this case not having breast cancer). Of this group, the biopsy results for 200 samples divided exactly into 50% non-disease and 50% having breast cancer disease and each group was further subdivided into specified age groupings.

The sample analysis results were used to develop a training set model that is predictive of the disease state. The remaining samples (about 110) were then processed as blinded samples through the training set model to obtain resultant cancer risk numerical scores and these scores were disclosed to the host clinical center. These blind sample scores subsequently were analyzed by the clinical center to assess the clinical accuracy of the results.

Two diagnostic models were developed for this experiment, and are referred in this specification as Algorithm I and Algorithm II. The Spatial Proximity method of analysis was used for both algorithms. The age of the subjects was not used as an independent variable but rather as a meta-variable to transform the measured concentrations into new independent variables, referred to in this specification as Proximity Scores, which were used directly in the correlation analysis. The difference between Algorithm I and Algorithm II is the number of new independent variables used in the correlation. Algorithm I uses five Proximity Score variables in a ten dimensional cluster space. The lower limit of Algorithm I is two dimensions and it is based not upon a specific method, but rather on the fact that a correlation is performed. A correlation inherently involves more than one dimension. The upper limit of Algorithm I is theoretically infinity but is practically limited by computing time and power. The cluster space can be viewed by the human eye via projection or cuts through this multidimensional space to look at a two-dimensional bi-marker plane. There are ten such planes in this exemplary embodiment of Algorithm I.

Algorithm II uses ten-fold more created independent variables, such that there are about 100 bi-marker planes. It is expected that 200 samples are sufficient for the training set model such that it reasonably closely models the general population. The secondary or the incongruent training set model was developed from the same 200 sample training data set. The training set model is the primary scoring method used to describe the results in this specification. The incongruent training set model is used to arbitrate primary training set model calculated cancer scores that are considered unstable; that is, scores that rest on an area of topological instability. Though the incongruent training set model is somewhat less accurate on blind samples, it still can arbitrate the primary training set model and thus improve predictive power.

The foregoing Spatial Proximity method of analysis has significant advantages relative to logistic regression, in that it is able to accommodate highly non-linear trends in the independent variables used to create the calculation outcome. The outcome is either disease or non-disease (in this case cancer or not cancer) and it is based upon the Proximity Scores to the training set model calculations. The disadvantage of this method is the highly non-linear areas can be associated with very steep topology slopes. Thus, an unknown (or blind) sample may be sitting on a steep peak or deep sharp valley, which has the effect of amplifying small errors in the computed Proximity Scores. We assessed the stability of the calculated scores with a proprietary stability test and then used Algorithm II to arbitrate Algorithm I for samples that showed stability.

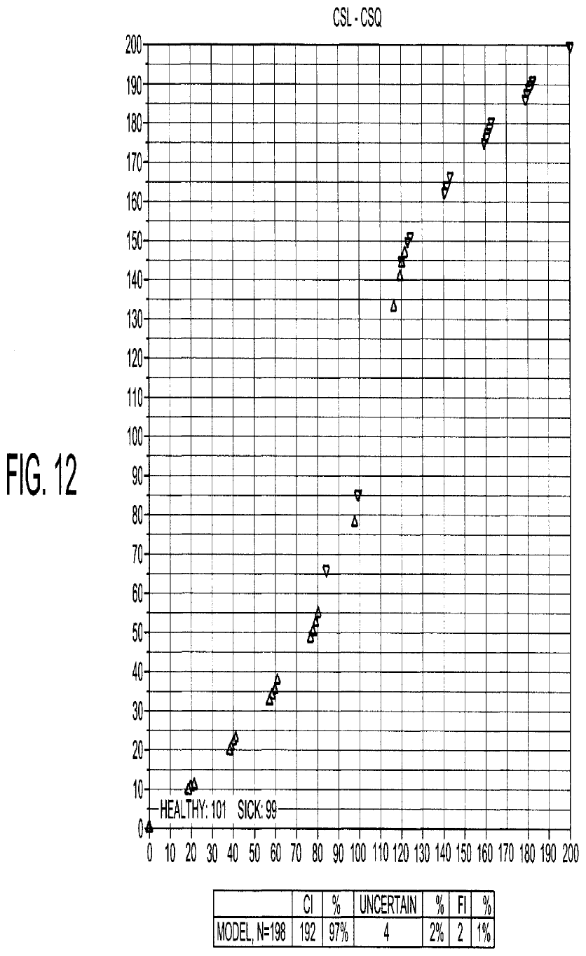

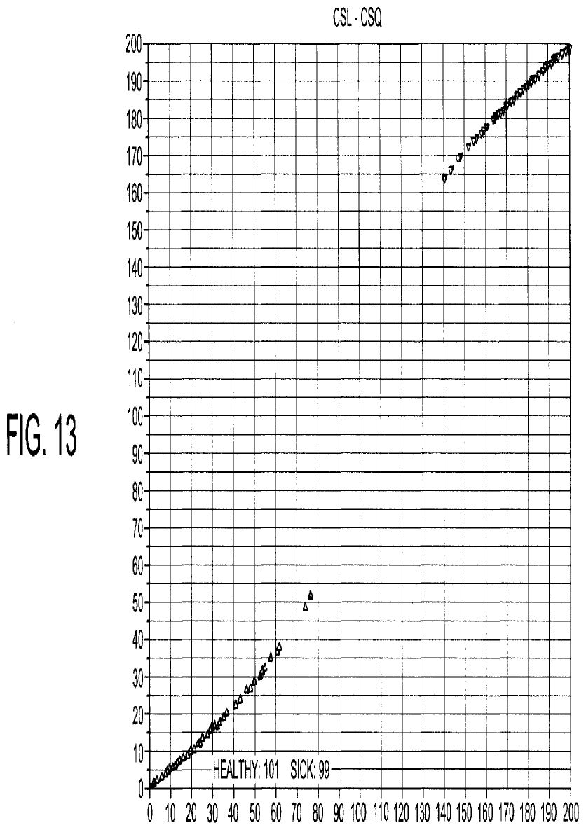

FIGS. 11, 12 and 13 show the Algorithm I training set results. The model itself consists of 10 bi-marker planes of 40,000 topology points each scored for non-disease and disease (here, breast cancer) by the Spatial Proximity method. The ability of the model to separate the two sets of non-cancer and cancer is shown in these figures. The model must be constructed from very close to or preferably exactly 50% by 50% or very close to one of the two outcome states. Also, the method uses age as a transforming meta-variable. The training set samples had samples distributed across all age groups of interest. Model (FIG. 12) for Algorithm I was constructed from 100 healthy women and 98 breast cancer women. The summary table in FIG. 12 shows the numerical results, where N=198 is the number of samples. CI is correctly called samples and FI is falsely called samples, and 4 samples were deemed uncertain. A secondary training set model was developed to discriminate the four uncertain samples that resulted from the use of the primary training set model. This model is the incongruent training set model. This secondary model uses the same training set data as the primary. FIG. 13 shows the results for the incongruent training set model calculations. Algorithm II shows 100% separation with over 60 points of separation.

Results of Testing Blind Samples in the Breast Cancer Study

FIG. 14 shows the results for the blind samples evaluated in the clinical study. The results show 100% sensitivity and 97.5% specificity. The oncologists at the clinical study center set the diagnostic transition value such that the breast cancer positive samples were all identified correctly. Thus, two non-disease samples were called positive for cancer. This is medically sound as the samples judged positive will all get the next diagnostic step, imaging mammography. Many women do not get imaging mammography because they do not live near enough to facilities with the medical equipment. However, their blood can be drawn remotely from the clinical lab and shipped on ice to a lab in a major city.

### EXAMPLE 2

### Use of the Meta-Variable “Age” to Improve Diagnostic Accuracy

Table 1 (below) shows the tabulated results for an 868 subject sample clinical study for breast cancer.

TABLE 1
Summary of Diagnostic Accuracy for Breast Cancer

| | | Correctly | | Falsely |
|---|---|---|---|---|
| Condition | Cohort | Identified | Uncertain | Identified |
| Breast Cancer | 495 | 98.0% | 1.0% | 1.0% |
| Healthy Women | 373 | 98.0% | 0.5% | 1.5% |

Table 2 (below) shows the comparison of various methods for the correlation calculation. The standard method, logistic regression, showed only an 82% predictive power. Standard Spatial Proximity analysis improved on this, yielding about 88% predictive power in linear form and 90% predictive power in logarithmic form. The methods described in this specification using the meta-variable and weighting approaches, topology stability conditioning, immune system response grouping and weighting conditioning for assay performance—coupled with instability testing of blind samples and incongruent algorithm correction—yielded greater than 97% predictive power.

TABLE 2
Comparative Predictive Power of Disease Correlation Calculations

| Method | | Predictive Power |
|---|---|---|
| Logistic Regression | | 82% |
| Spatial Proximity Analysis | Linear | 88% |
| | Logarithmic | 90% |
| Meta-Variable method | | >95% |

### EXAMPLE 3

### Use of the Meta-Variable “Age” to Improve Diagnostic Accuracy in an Ovarian Cancer Study

Table 3 (below) shows the results of a study of 107 women with ovarian cancer or not having ovarian cancer using the meta-variable method described in the embodiments herein. This study did not use all of the predictive power improvements described in this specification but still achieved a relatively superior predictive power of about 95%.

TABLE 3
Summary of Diagnostic Accuracy for Ovarian Cancer

| | | Correctly | | Falsely |
|---|---|---|---|---|
| Condition | Cohort | Identified | Uncertain | Identified |
| Ovarian Cancer | 51 | 94.1% | 3.9% | 0.0% |
| Healthy Women | 56 | 96.4% | 3.6% | 0.0% |

### EXAMPLE 4

### Use of the Meta-Variable “Age” to Improve Diagnostic Accuracy in Prostate Cancer

Table 4 (below) shows the results of a study of 259 men either having prostate cancer or benign prostate hyperplasia (BPH) using the meta-variable method described in this specification. This study also did not use all of the predictive power improvements described herein but still achieved a relatively superior predictive power of about 94%. Note that BPH is by far the most common condition that causes false positive results in the current PSA test for prostate cancer. Men with BPH are about 4 out of five positives in conventional diagnoses of prostate cancer resulting in most prostate cancer biopsies being negative. The meta-variable method is able to correct these incorrect diagnoses as discussed above.

TABLE 4
Summary of Diagnostic Accuracy for Prostate Cancer

| | | Correctly | | Falsely |
|---|---|---|---|---|
| Condition | Cohort | Identified | Uncertain | Identified |
| Prostate Cancer | 111 | 93.70% | 0.90% | 5.40% |
| Benign Prostate | 148 | 95.90% | 0.00% | 4.10% |
| Hyperplasia/Hypertrophy | | | | |

The foregoing results in Examples 3 and 4 (for ovarian cancer and prostate cancer, respectively), did not use the meta-variable or influence adjustment methods (LOD, sub-populations groupings and instability) nor the blind sample stability method.

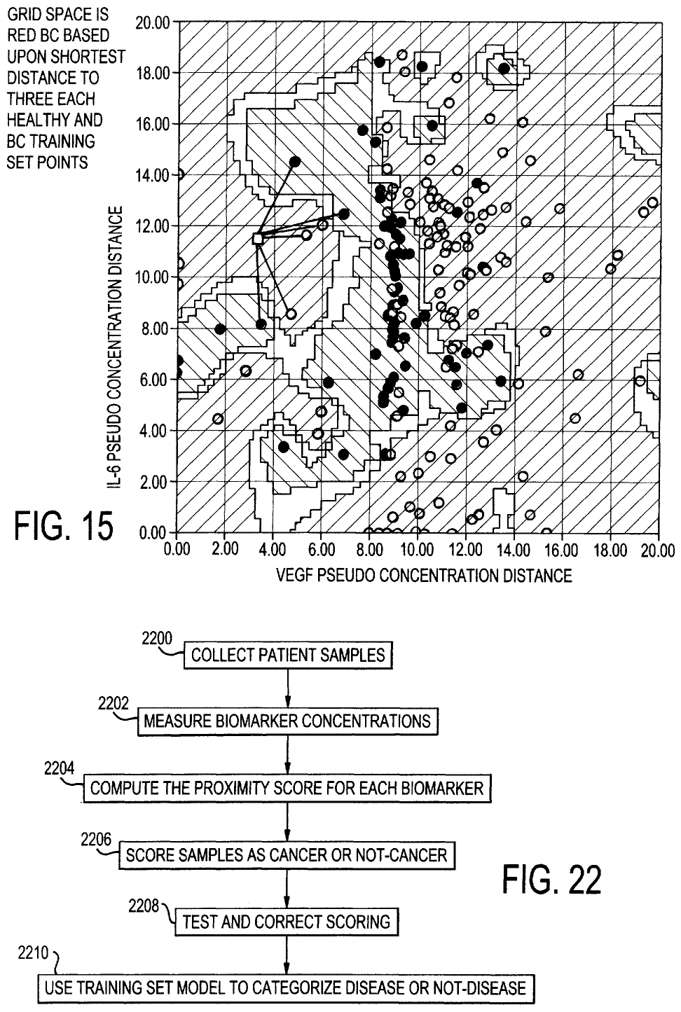

In order to further improve predictive power, these age or grouping-adjusted concentrations are conditioned to normalize them and reduce or eliminate spacing bias (also known as spatial bias) in the clustering across the multidimensional grouped marker plots for the Spatial Proximity analysis. See for example, FIG. 15, which presents the bi-marker plane for IL-6 and VEGF. There are ten of these planes for the five-biomarker breast cancer test panel. In this case, the calculated Proximity Score values are normalized and shifted to produce arbitrary values between zero and twenty with outlier highly up-regulated concentrations being highly compressed.

Each of the bi-marker projections of the multi-dimensional marker planes on the same normalized spacing over the concentrations from the age/grouping analysis are compressed and normalized against the age adjusted means as well as age (or whole populations) adjusted sub-groupings.

Improvements in Predictive Power of the Training Set Model Using Adjustable Bi-Marker Plane Influence Levels

Typically, the bi-marker plane will be scored with binary numbers for non-disease and disease (for example, +1, and -1). The Proximity Score method described herein is amenable to further improvements in predictive power by selectively adjusting the influence levels of these two binary numbers. The methods below are developed in the training set model and once set are fixed in the model.

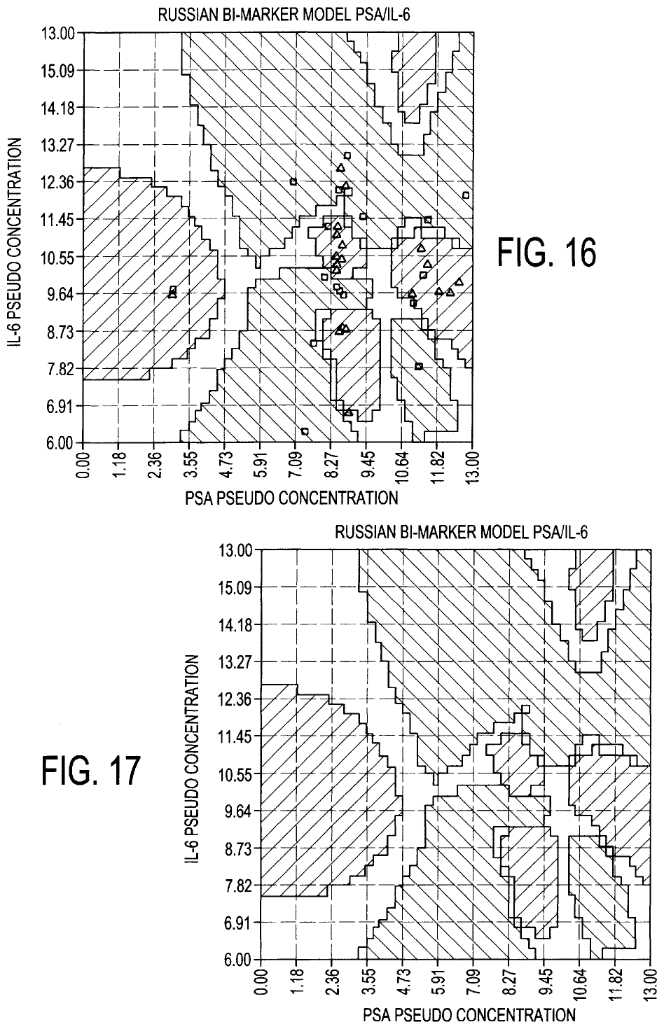

FIGS. 16 and 17 show the projections of one bi-marker plane for the case of five biomarkers used to predict presence of the disease state, in this case breast cancer using the five markers; IL-6, IL-8, TNFα, VEGF and PSA. FIG. 16 shows the training set model with the data used to score the grid points on the plot by the Spatial Proximity analysis method. FIG. 17 shows the training set model without the data. This constitutes the training set model. The training set data used for creating the model are not needed as each of the 40,000 grid points are scored and a blind sample is scored by where it lands on the grid. The topology shows red positive for cancer and the blue are negative for cancer. In computing the overall score in this case, the non-disease grid points are set at +1 and the disease (cancer) grid points are set at −1. Each bi-marker in this five-biomarker example is analyzed in a five orthogonal space of which FIG. 16 is one projection of two dimensions. On this plot are shown the topology of the various sub groupings of immune system response. In this case, the all grid spots (2000 ×2000 or 40,000 in this case) are scored in the usual way and the value assigned is −1 for disease state positive (breast cancer) and non-disease is +1. This bi-marker plane is normalized by Proximity Score spacing and for the meta-variable age as noted above.

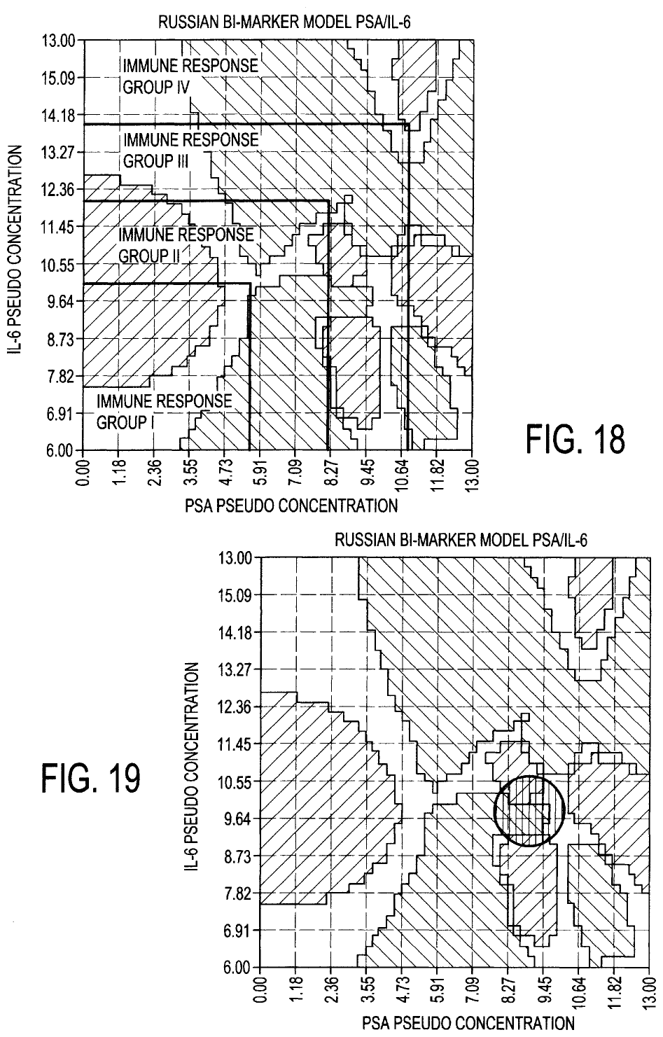

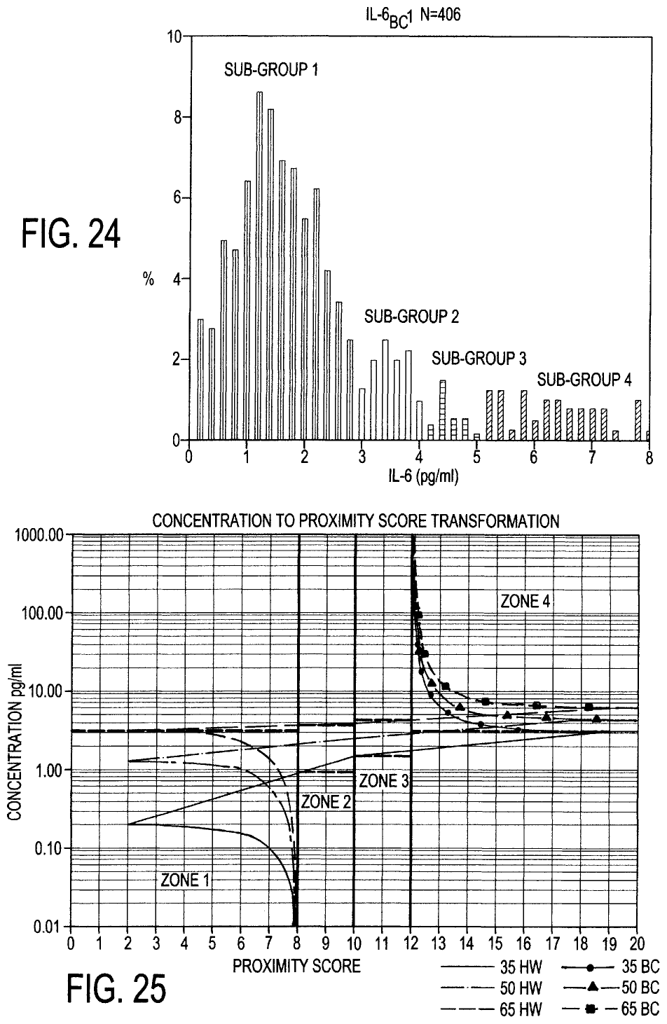

FIG. 18 shows the same bi-marker model and additionally the immune response groupings (see FIG. 24) inside the grey areas. The grayed areas influence is adjusted to reflect the fact that each grey blocked area has a somewhat different influence on the probability that the patient is non-disease or disease. This adjustment can be made either by human estimate with training set validation, or by rigorous computer multi-variable incremental analysis. These adjustments improve the training set model. Two separate bi-marker planes are created for the two outcomes, which are the disease and non-disease states. In this case, blind data points in the Immune Response Group IV are much more likely to be disease and the influence would be increased (absolute value) slightly (for example, by changing the score from −1 to −1.1). The actual amount of this increment preferably would be determined by computer analysis or possibly by rigorous manual methods. This method is workable for the Spatial Proximity (also known as pseudo-concntration)method of correlation analysis but other means could be used to the same effect. These methods of weighting the influence with respect to association of disease can produce an improvement in predictive power of about 1%. At predictive powers above 95% this is very significant.

FIG. 19 shows again the same bi-marker plane with a grey area circled in a complex area of non-linear, rapidly changing disease vs. non-disease topology. Such areas can be identified by inserting test blind sample values with injected noise (say +/−10%) into the model and then injecting a measured amount of noise. Most of these blind points will not change substantially in disease (here, cancer) score. Some grid points, however, may be found that jump dramatically from a non-disease to disease score after this kind of noise adjustment. These are areas where most or all of the bi-marker planes have rapidly changing topology that overlaps the multi-dimensional overall bi-marker planes. By careful reduction in influence in these areas, weighting can be increased in the few relevant bi-marker planes that the noisy datum sits on a broad plane without being near changing outcome boundaries. This method has been shown to correct erroneous predictions. In the case above, the influence of the red, cancer areas would be shifted down (absolute value), for example, from -1.0 to -0.9. Or the blue non-disease areas would be shifted down from +1.0 to -0.9. The level of optimal shift could be determined by rigorous computer analysis.

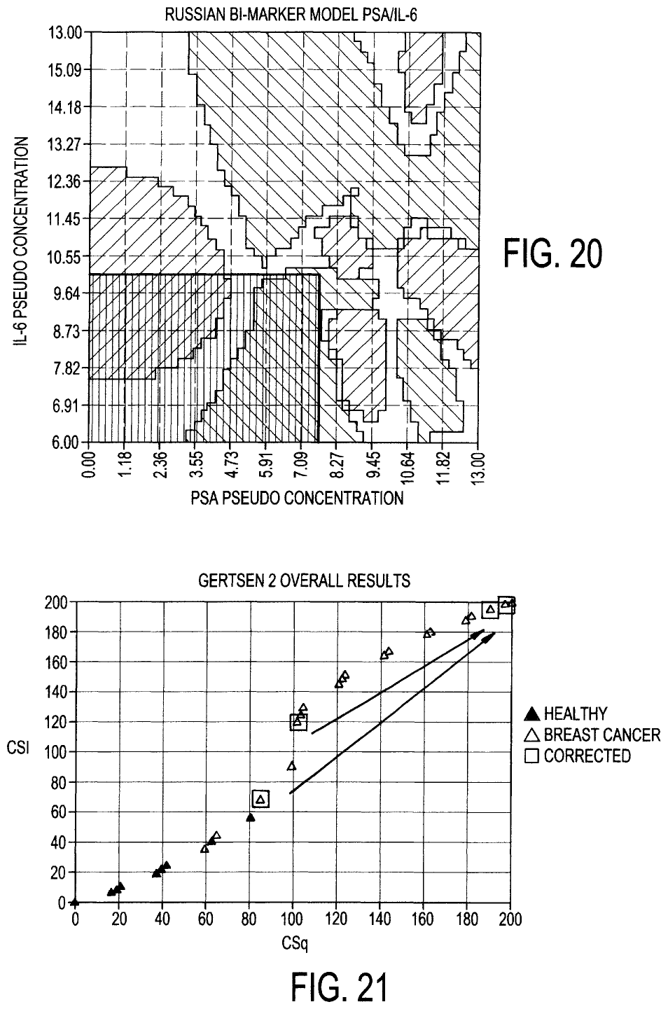

Assay noise can affect the accuracy of the correlation analysis. This noise can be especially problematic at levels at or below the assay's limit of detection. This noise also can be mitigated by reducing the influence of measured points for individual biomarkers that are in these unstable zones. FIG. 20 again shows the bi-marker plane for PSA and IL-6 for a breast cancer panel. Areas within the grayed rectangular area at the bottom left of the figure are all below the traditional limit of detection (LOD) of the assay. Traditionally LOD is defined as two standard deviations of 20 zero calibrators plus the average of the value of the twenty zero calibrators. The statistical certainty for the values at this level are 95% within two standard deviations, and of course the measurement certainty goes down as the measured sample goes lower than the LOD. The data still may still have useful information but should be applied to the analysis with less influence. In this case, the influence on blind sample datum points within the grayed area are reduced, for example, from +1.0 to −0.9 for grid points of the training set model within the gray area. This increases the influence for datum points for this test sample that are above the limit of detection on their, other bi-marker planes. The foregoing methods are complimentary and can implemented in tandem.

Methods for Improving Predictive Power by Testing the Blind Samples for Instability

Once the training set model is complete and fixed, it is used to calculate cancer scores for blind patient samples. The inventors use two preferred methods for producing cancer scores. The first, termed the linear method (CS1) takes the topology location score (+1 or -1) multiplied by the predictive power for that bi-marker plane. These are then added up and scaled and shifted to yield a score from 0 to 200. The second score, termed the q score (CSq) is calculated by using the square root of the sum of the squares on these same values. This second method accentuates differences in individual bi-marker scores and is useful in the overall physician's ultimate diagnosis.

Topology instability does still remain in the bi-marker planes due to the highly non-linear nature of the Spatial Proximity method of correlation and cannot be completely eliminated. According to other aspects of the present invention, a stability test and techniques involving injected noise can be applied to the blind data set. And an incongruent training set model can be used to arbitrate or correct cancer scores. For this aspect of the invention, a fixed level of noise is injected for each blind patient data set (for example, plus or minus 10%). If the blind sample set is about 100 patients, then the actual training set model computer run will be for 300 samples set with each in triplicate (the raw data plus noise and minus noise). The resulting triplicate data set are then tested for stability (a is −10%, b is +10% and the c point is the raw data). Table 5 (below) shows the result of the stability test for data from the clinical study. Notice that three samples show very high instability in the cancer scores. Samples 138, 207, 34 and 29 all show very high figure of merit. The figure of merit (lower better) should encompass both the degree of score shifting and especially whether or not the score shifts for predicting healthy to cancer or vice versa. These data sets from blind samples are at a high risk of being incorrect in predicted diagnosis.

TABLE 5
Results of Topology Instability Test

| Sample | Age | CSL | CSQ | Stability Figure of Merit |
|---|---|---|---|---|
| 138a | 68 | 104.375 | 129.943 | 438 |
| 138b | | 43.244 | 26.996 | |
| 138c | | 85.23 | 69.261 | |
| 207a | 70 | 82.546 | 61.476 | 24039 |
| 207b | | 82.546 | 61.476 | |
| 207c | | 142.396 | 166.928 | |
| 29a | 69 | 79.4 | 53.971 | 351 |
| 29b | | 161.089 | 178.912 | |
| 29c | | 161.089 | 178.912 | |
| 34a | 67 | 102.019 | 120.426 | 853 |
| 34b | | 59.671 | 36.265 | |
| 34c | | 102.019 | 120.426 | |
| 65a | | 200 | 200 | 22 |
| 65b | | 180.581 | 190.11 | |
| 65c | | 180.581 | 190.11 | |
| 72a | | 181.757 | 191.335 | 11 |
| 72b | | 181.757 | 191.335 | |
| 72c | | 181.757 | 191.335 | |
| 74a | | 141.818 | 166.105 | 95 |
| 74b | | 141.649 | 165.886 | |
| 74c | | 121.016 | 147.092 | |
| 77a | | 181.073 | 190.646 | 36 |
| 77b | | 161.759 | 179.731 | |
| 77c | | 181.073 | 190.646 | |
| 80a | | 141.398 | 165.473 | 64 |
| 80b | | 161.395 | 179.26 | |
| 80c | | 141.398 | 165.473 | |
| 286_1a | | 19.165 | 9.629 | 36 |
| 286_1b | | 38.382 | 20.474 | |
| 286_1c | | 19.165 | 9.629 | |
| 287_1a | | 0 | 0 | 0 |
| 287_1b | | 0 | 0 | |
| 287_1c | | 0 | 0 | |
| 288_1a | | 0 | 0 | 0 |
| 288_1b | | 0 | 0 | |
| 288_1c | | 0 | 0 | |
| 289_1a | | 0 | 0 | 0 |
| 289_1b | | 0 | 0 | |
| 289_1c | | 0 | 0 | |
| 290_1a | | 0 | 0 | 0 |
| 290_1b | | 0 | 0 | |
| 290_1c | | 0 | 0 | |
| 291_1a | | 0 | 0 | 0 |
| 291_1b | | 0 | 0 | |
| 291_1c | | 0 | 0 | |
| 245_1a | | 0 | 0 | 124 |
| 245_1b | | 81.565 | 58.963 | |
| 245_1c | | 40.353 | 22.961 | |
| 246_1a | | 42.152 | 25.392 | 25 |
| 246_1b | | 42.152 | 25.392 | |
| 246_1c | | 42.152 | 25.392 | |

An incongruent training set model can be used to arbitrate “at risk” patient sample data sets that fail a merit noise test. These points are at risk due to inevitable measurement noise, either random or systematic coupled with extreme topology instability caused by the fact that the blinded sample data point sits on a very steep slope on most if not all of the bi-marker planes so that small perturbations yield large swings in score. Table 5 shows samples with noise injected. Each sample has three values, 1) plus noise, 2) minus noise and 3) raw data no noise. These samples show cancer scores that jump from disease to non-disease and back with the injection of +−10% noise. These sample data in this case are judged to be unstable. The level of instability is not exactly defined and adjustments can be made for various levels of noise injection. In this case, these are corrected with +−10% noise and a stability score of greater than 200 (note that stability score and cancer score are two distinctly different number with different meanings).

Measurement noise can be arbitrated with this incongruent second algorithm (Algorithm II). The incongruent algorithm used for arbitration can be used to correct these “at risk” patient samples sets even if it has slightly less predictive power than the main algorithm as it will improve the odds that the point is correct. In this case, two were corrected (see FIG. 21); sample 138 had a score of 85 non-disease and was corrected to 195 with the incongruent algorithm (this point was stable with Algorithm I, sample 34 had a score of 102 (linear method) and was corrected to 198 again with Algorithm II. Samples 29 and 207 were not changed by the incongruent algorithm.

The incongruent training set model (Algorithm II) used 105 bi-marker planes and is incongruent to the primary training set model (Algorithm I) in that these same samples show as stable in the Algorithm II stability test. Testing the incongruent training set model is done in exactly the same way as for the primary training set model. Note that the logistic regression method could be also used to calculate these sample scores. Algorithm II has a high predictive power so it was used. An arbitrating training set model can be used even if its predictive power is less (preferably, not less than 50% predictive power though) than the main algorithm as long as it has a likely correct result without instability. Notice that the correction is dramatic for the blinded samples in question that failed the noise test. These samples actually were all cancer with high scores. Eight of the ten bi-marker planes for these samples were on topology with very high unstable grid points. Thus the scores were at risk and indeed were incorrect (one was incorrect and one was uncertain with scores of 100/120). In this case, one sample was corrected to improve the predictive power from 97% to 98%, a very significant reduction in error (50%). One sample, though uncertain, was changed to cancer and also corrected.

Method for Improving Disease State Correction Binary Outcome Predictive Power by Excluding an Independent State that Partially Mimics One of the Outcome States of the Primary Disease Analysis

Spatial Proximity analysis commonly uses three or more independent variables, often a patient's blood serum protein concentrations. The correlation algorithm can act on only a binary outcome of non-disease or disease, but it produces a continuous scoring that more closely relates to a probability of the actual outcome being the two binary conditions. In some cases, there are other conditions, nominally classified as non-disease, that partially mimic the disease state within the population distributions of the biomarkers used. In some of these cases, this non-disease

“MIMIC” state can cause a false positive outcome of the correlation analysis. A solution to resolve this kind of false positive result is to create an additional new correlation analysis completely separate from the non-disease or disease analysis. This new correlation analysis preferably uses the exact same biomarker measured data as for the non-disease or disease correlation or it may use some or all different biomarkers. This new correlation analysis provides a result of “non-disease MIMIC” or “disease” or at least produces a score allowing a judgment to be made about the real state of the patient. An uncertain or near transition score for the non-disease or disease analysis coupled with a very low or high score in the non-disease MIMIC or disease correlation can help the physician practitioner improve the disease state judgment and reduce false positive scores.

An example of this situation where a non-disease condition mimics a disease state is the non-malignant condition Benign Prostate Hypertrophy (BPH). This condition will commonly show high levels of at least one biomarker used to diagnose prostate cancer. For example, the biomarker, prostate specific antigen (PSA), will be elevated in men with BHP and also with prostate cancer. Table 4 shows that this additional correlation analysis method can discriminate between men with BHP and prostate cancer and, likewise using the same biomarkers but a different training set model, can discriminate between men who are putatively in a non-disease state and those with confirmed prostate cancer in the disease state. In a small fraction of men, a false positive will result with the non-disease versus cancer training set model, but this will be discriminated by the BHP versus cancer training set model. In these cases, two scores, one for putatively non-disease verses cancer and one for BHP verses cancer, will help the physician or other health care practitioner decide the next diagnostic step. For example, for total scoring (for either CS1 or CSq) from 0 to 200 for both models, a score of 110 for “NOT PROSTATE CANCER OR PROSTATE CANCER” indicates a weak score for being cancer positive but also considering the second score of 30 for the BPH or cancer would indicate to the physician practitioner a high likelihood of BPH not cancer. The physician practitioner would use this added information along with other medical information and patient history to decide the next steps in diagnosis.

Detailed Discussion of the Methods

Analytical Steps/Algorithm

The process for developing an analytical model in accordance with the present invention generally follows the logic pathway described below, as exemplary shown in FIG. 22:

At step 2200, COLLECT PATIENT SAMPLES, the software will collect a large group of known not-disease and disease patient samples. The samples are generally not screened for any other unrelated conditions (non-malignant for cancer) but collected such that the sample sets look statistically like the general population.

At step 2202, MEASURE BIOMARKER CONCENTRATIONS, the software measures the biomarker parameter concentrations using methods and devices known in the art.

At step 2204, COMPUIE THE PROXIMITY SCORE FOR EACH BIOMARKER, the software computes the Proximity Score curves for each biomarker and sets the zones for each, as shown in FIG. 25.

At step 2206, SCORE SAMPLES AS CANCER OR NOT-CANCER, the software runs the model program to score the samples using the Spatial Proximity Correlation Method. The model uses compression or renormalization equations unique to each of the 4 zones (see Equation 1 below).

At step 2208, TEST AND CORRECT SCORING, the software tests individual samples for topology stability and correct those that fail with the incongruent algorithm. First, all cancer scores are tested for topology stability in the usual way by injecting a plus minus noise on the measured concentration level, computing the dithered proximity scores and applying these to the primary Spatial Proximity Model. If these dithered cancer scores shift beyond a predetermined limit, the computed cancer score using the primary model is rejected. The original concentration levels for the failed tests are then transitioned to new proximity scores using the secondary or incongruent model. These new Proximity Scores for these failed samples are then applied to the Spatial Proximity Correlation model. These new cancer scores are then tested with the secondary model for stability in the same way. If these samples pass the stability test, then they are reported as having been analyzed by the incongruent model. If both the primary and secondary model are unstable, the sample will be reported as uncertain.

Finally, at step 2210, the software outputs the above-discussed results at TRAINING SET MODEL TO CATEGORIZE DISEASE OR NOT-DISEASE.

Devices and Reagents Used for Breast Cancer Validation Study: Test Platform Description

OTraces CDx Instrument System

The test data included below and for much of the work discussed above was measured on the devices and with the reagents noted below. The data was processed on the OTraces LIMS system, or in some cases calculations were completed on PC-based software. All of the computational software was written and validated by OTraces Inc. It will be readily apparent to one of ordinary skill in the art that other equivalent hardware, devices, and reagents may be used to achieve similar results.

The CDx Instrument System is based upon the Hamilton MicroLab Starlet system. It is customized with programming to transfer the OTraces immunoassay methods to the Hamilton high speed ELISA robot. The Hamilton Company is a well-respected company that sells automated liquid handling systems worldwide, including the MicroLab Starlet. The unit is customized by Hamilton for OTraces to provide for full automation. OTraces CDx System includes an integral Microplate Washer System and Reader. These two additional devices allow the system to complete one full run of all five immunoassays in the test panel in one shift with no operator intervention after initial setup. The system as configured will complete 40 cancers scores per day. Enhancements include software to conduct one target analyte at a time. This is needed to be able to rerun a specific test when an error occurs within a full test run.

BC Sera DX Test Kit

This test kit includes all of the reagents and disposable devices to perform 120 cancer test scores, including all buffers, block solutions, wash solution, antibodies and calibrators. Enhancements needed to fully commercialize this test kit include adding two control samples. These controls provide independent validation that a “blind” test sample yields a proper cancer score. The two controls are designed to produce a Proximity Score of 50 and 150 respectively. The LIMS system (see below) QC program will verify that these controls are correct, thus validating the individual test runs in the field. The test kits are built in a GMP factory and have received the CE mark. The microtiter plates are pre-coated at the factory with the capture antibody and protein blocking solutions.

Laboratory Information Management System (LIMS)

Clinical chemistry systems marketed today, e.g. by Roche and Abbott, all include a graphical interface with software sufficient to manage patient data, quality control the instrument and chemistry operations and facilitate test sample identification and introduction to the test system. These menus are integrated into the delivered chemistry system. OTraces' business model is to include these functions on OTraces computer servers located at OTraces' US facilities and connect the CDx instrument integrally to these servers through the Internet using cloud computing. This yields several significant advantages: 1) The LIMS software incorporates FDA compliant archival software such that data from all test runs from each CDx system deployed in the field are run on the OTraces servers. Applying feedback from the installed base, input from key institutions about patient outcomes allows OTraces to collect FDA compliant data for US based FDA market clearance submissions. 2) Preferably, bar coded reagent packaging allows the instrument and LIMS to connect all QC test results from the factory QC test. These data are available in real time as the tests are run in the field for further validation of the field test results. 3) The CDx System will only run OTraces validated reagents and thus test runs using non OTraces reagents will not be possible. This system appears as a typical user interface to the operator with all functions running in real time and patient reports are available as soon as the test run is complete.

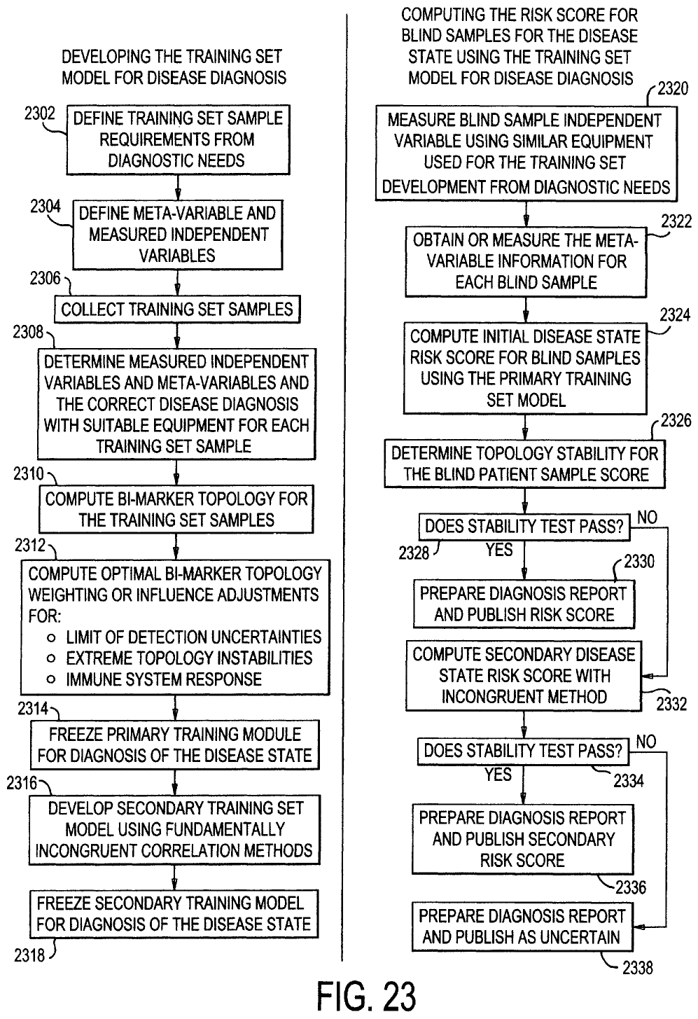

The stepwise process for developing a training set model and computing a risk score is shown in the flow chart of FIG. 23. This process may be implemented in software in certain embodiments of the invention. Construction of the Training Set Model is done first and its end product enables producing diagnostic results for unknown patient samples, termed blind samples, as the correct diagnosis is not known at the time of analysis for these blind samples. In general, the present invention provides a risk score to a health care provider who then considers this score along with other patient factors to make a medical judgment about the presence or absence of a given disease state.

Steps 2302 through 2318 outline the process by which the training set model is created. At step 2302, the software defines the training set sample requirements from diagnostic needs, which are predetermined criteria that may be set by one of ordinary skill in the art. For example, these criteria may be a disease vs. non-disease state, more specifically, for example, breast cancer, comparing breast cancer positive vs. samples known to not have breast cancer

At step 2304, the software defines the meta-variables to be calculated as well as the independent variables (i.e. biomarkers) to be measured.

At step 2306, the software collects the training set samples in accordance with the parameters set in steps 2302 through 2304. At step 2308, the software determines measured independent variables and meta-variables, as well as the correct disease diagnosis associated with those results, using suitable medical equipment for each training set sample. At step 2310, the software computes bi-marker topology for each of the training set samples. At step 2312, the software computes optimal bi-marker topology Weighting or influence adjustments for the following: (1) Limit of Detection Uncertainties, e.g samples that are determined to be below classical limit of detection; (2) Extreme Topology Instabilities, e.g. as determined by methods described in ¶ [0111] and with respect to the topology stability discussion above. At step 2314, the calculations are considered complete and the primary training model is frozen for diagnosis of the disease (for example, cancer). At step 2316, the software develops a secondary training model using fundamentally incongruent correlation modeling (see, for example, FIG. 10). At step 2318, the calculations are considered complete and frozen, as the secondary training model for diagnosis of the disease state is created. In this manner, a training model set is created for diagnosing the disease.

Steps 2320 through 2338 describe how the software of the present invention uses the training model developed to diagnose diseases like cancer. At step 2320, the software measures blind sample independent variables like biomarkers using medical equipment similar to that used in the development of the training set model. At step 2322, the software obtains or measures and calculates meta-variable data for each blind sample. At step 2324, the software uses that data to compute an initial disease state risk score for the blind sample using the primary training set model. At step 2326, the software determines the topology stability of the blind patient sample score. At step 2328, the software checks whether the score passes the topology stability test. The criteria for pass/fail entails determining how large the instability induced error is and most importantly whether the score flips from disease positive to negative or vice versa. At step 2330, if the score is found to pass the stability test, a diagnosis report and risk score are output and/or published. If the score does not pass, then at step 2332, the software further computes a secondary disease state risk score using the incongruent method algorithm (Algorithm II) described above. At step 2334, the software again checks whether the score passes the topology stability test. At step 2336, if the score is found to pass the stability test, a diagnosis report and risk score are output and/or published. In the score still does not pass, at step 2338, the software prepares a diagnosis report and outputs and/or publishes the results as uncertain as to whether a disease state exists.

Proximity Scores, as computed herein, have several unique properties. In certain embodiments, the mean values of the proteins are embedded in the logarithmic compression as a ratio to the actual measured concentration for the patient with that age. In essence, the method creates a fan of similar equations that are each unique to, for example, the age in years of the patient population. Each unknown sample gets a unique equation for the sample's age.

A relationship that includes an age adjusted mean for non-disease and disease and the actual patient sample concentration of the following form can be used:

Proximity Score=(K)*In ((Cᵢ/C_{(c or h)})−(Cₕ/C_{c}))² where: EQUATION 1

K=proportional constant;

Cᵢ=measured concentration of the actual patient's analyte;

C_{(c or h)}=patient age adjusted concentration of this patient analyte; the value is adjusted for whether the patient is a non-disease or disease state;

Cₕ=patient age adjusted mean concentration of non-disease patients' analyte; and

C_{c}=patient age adjusted mean concentration of disease patients' analyte.

This Equation 1 is designed to adjust compression and expansion depending on the up-regulation grouping zone, as shown in FIG. 25. The formula above for Proximity Score accomplishes this requirement; however, many other forms of this equation can be implemented as will be apparent to persons skilled in the art. For example, Cᵢ, Cₕ and C_{c} could be actual concentrations or concentration distances from the mean, medium or distance from sub group medians or dynamic range edges as discussed above. Other variations of this calculation are reproduced below as Equations 2 and 3.

Proximity Score=K* ln (((concentration of unknown sample)/(Concentration of mean value of cancer at age of unknown sample))−((Concentration of mean value of not cancer at age of unknown sample)/(Concentration of mean value of cancer at age of unknown sample)))². EQUATION 2

This equation yields negative infinity (natural log of zero) when the unknown sample is equal to the not-cancer mean at the unknown samples age. This is overridden with the actual detail equation to a set value, for example, 2, as shown in FIG. 25. In other words, values outside of the preset range are tested and reset by the software to the value at the limit of the preset range.

Proximity Score=K*ln (((concentration of unknown sample)/(Concentration of mean value of not cancer at age of unknown sample))−((Concentration of mean value of cancer at age of unknown sample)/(Concentration of mean value of not cancer at age of unknown sample)))². EQUATION 3

Equation 3 yields negative infinity natural log of zero when the unknown sample is equal to the cancer mean for that unknown sample's age. This embodiment of the equation is used when the unknown sample is above the midpoint concentration between the not cancer and cancer means at the unknown sample's age (putatively cancer). In this situation the whole equation is inverted thus to positive infinity when the unknown sample is at the mean value of cancer for its age. This infinity is overridden within the actual detail equation to a set value, for example, 18. The graph in FIG. 25 shows the family of equations that result for the age range of interest. The equations operate on each of the four zones shown in FIG. 25 independently. The zones are: 1) below the mean value for the not disease population; (2) above the not disease mean value and below the derived midpoint between the not disease and disease mean value (the not disease/disease transition); (3) between the derived midpoint between the not disease/disease mean value and the population disease mean value; and (4) above the mean value for the disease state. Note that these zones do not indicate the samples located within the zone is that disease or not disease state. An individual sample's true diagnosis may be either and its position if “incorrect” may be because of another condition that effects the biomarker. We term this proteomic noise. The zones simply denotes how the individual samples relates to the means, and provides a scaffolding for the compression or renormalization done by exemplarily by Equation 1. Note that each equation represents only one age value and that the overall set constitutes a multiplicity of equations each that represent a single age value. The overall set of equations are designed to set the Proximity Score values at the same predetermined value for all ages when the actual concentration equals exactly the mean values. The ages shown are 35, 50 and 65. The full set looks like a fan, with one equation for each unknown sample age.

Proximity Scores (unit-less and thus not concentrations or levels) are exemplarily calculated as described above and are then used in the Spatial Proximity correlation multidimensional plot for analysis. Also, all of the plots are normalized to common characteristics of the population distribution; age mean values of non-disease and disease (age adjusted or not), median value, or dynamic range of sub groupings. These methods can yield improvements in predictive power of 5 or more percentage points.

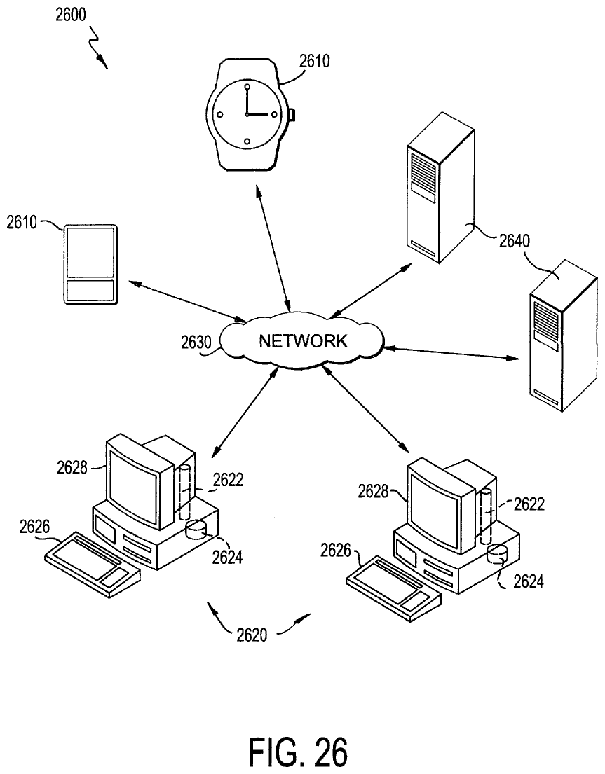

The foregoing exemplary embodiments of the systems and methods of the present invention may be implemented in software running on both networked and non-networked hardware. An exemplary embodiment of the hardware used to implement the invention is described in connection with FIG. 26. In the exemplary system 2600, one or more peripheral devices 2610 are connected to one or more computers 2620 through a network 2630. Examples of peripheral devices 2610 include smartphones, smartwatches, tablets, wearable electronic devices, medical devices such as EKGs and blood pressure monitors, and any other devices that collect biomarker data that are known in the art. The network 2630 may be a wide-area network, like the Internet, or a local area network, like an intranet. Because of the network 2630, the physical location of the peripheral devices 2610 and the computers 2620 has no effect on the functionality of the invention. Both implementations are described herein, and unless specified, it is contemplated that the peripheral devices 2610 and the computers 2620 may be in the same or in different physical locations. Communication between the hardware components of the system may be accomplished in numerous known ways, for example using network connectivity components such as a modem or Ethernet adapter. The peripheral devices 2610 and the computers 2620 will both include or be attached to communication equipment. Communications are contemplated as occurring through industry-standard protocols such as HTTP.

Each computer 2620 is comprised of a central processing unit 2622, a storage medium 2624, a user-input device 2626, and a display 2628. Examples of computers that may be used are: commercially available personal computers, open source computing devices (e.g. Raspberry Pi), commercially available servers, and commercially available portable device (e.g. smartphones, smartwatches, tablets). In one embodiment, each of the peripheral devices 2610 and each of the computers 2620 of the system may have the software related to the system installed on it. In such an embodiment, biomarker data may be stored locally on the networked computers 2620 or alternately, on one or more remote servers 2640 that are accessible to any of the networked computers 2620 through a network 2630. In alternate embodiments, the software runs as an application on the peripheral devices 2610.

While certain features of the described implementations have been illustrated as described herein, many modifications, substitutions, changes and equivalents will now occur to those skilled in the art. Thus, it will be understood that the invention is not limited to the particular embodiments or arrangements disclosed, but is rather intended to cover any changes, adaptations or modifications which are within the scope and spirit of the invention as defined by the appended claims. All references cited herein, including patents and applications, are expressly incorporated in their entirety.

1. A computer-implemented method for diagnosing a disease comprising the steps of:
(a) receiving a first set of one or more concentration values of a first biomarker from a first patient sample, wherein the first patient sample is comprised of not-disease diagnoses;
(b) receiving a second set of one or more concentration values of the first biomarker from a second patient sample, wherein the second patient sample is comprised of disease diagnoses;
(c) computing a first set of Proximity Scores from the first set of concentration values and a second set of Proximity Score from the second set of concentration values; and
(d) computing a correlation for the first biomarker to a disease diagnosis from the first and second set of concentration values and the first and second set of Proximity Score values, wherein the correlation is one of a simple regression, an ROC curve area maximization, a topology stabilization, or a Spatial Proximity analysis.

2. The computer-implemented method of claim 1, wherein steps (a)-(d) are repeated for up to five biomarkers.

3. The computer-implemented method of claim 1, wherein the correlation combines two or more of the simple regression, the ROC curve area maximization, the topology stabilization, and the Spatial Proximity analysis.

4. The computer-implemented method of claim 1, wherein the first and second patient samples include at least one of blood samples, urine samples, or tissue samples.

5. The computer-implemented method of claim 1, wherein the disease diagnosed is one of prostate cancer, breast cancer, lung cancer, or ovarian cancer.

6. The computer-implemented method of claim 5, wherein the disease diagnosed is the stage of the prostate cancer, breast cancer, lung cancer, or ovarian cancer based on Gleason Score.

7. The computer-implemented method of claim 6, wherein the first and second patient samples comprise cancer stage data, and wherein the cancer stage data is categorized into a plurality of binary groups.

8. The computer-implemented method of claim 7, wherein each of the binary groups is scored.

9. The computer-implemented method of claim 1, wherein the biomarkers are selected from a functional group of cytokines, and wherein the functions of the cytokines are at least three of: pro-inflammatory, anti-inflammatory, anti-tumor genesis, cell apoptosis, and vascularization.

10. The computer-implemented method of claim 1, wherein the first biomarker is VEGF.

11. A non-transitory computer-readable medium that stores a program thereon that causes a computer to execute a process comprising:
(a) receiving a first set of one or more concentration values of a first biomarker from a first patient sample, wherein the first patient sample is comprised of not-disease diagnoses;
(b) receiving a second set of one or more concentration values of the first biomarker from a second patient sample, wherein the second patient sample is comprised of disease diagnoses;
(c) computing a first set of Proximity Scores from the first set of concentration values and a second set of Proximity Score from the second set of concentration values; and
(d) computing a correlation for the first biomarker to a disease diagnosis from the first and second set of concentration values and the first and second set of Proximity Score values, wherein the correlation is one of a simple regression, an ROC curve area maximization, a topology stabilization, or a Spatial Proximity analysis.

12. The non-transitory computer-readable medium of claim 11, wherein steps (a)-(d) are repeated for up to five biomarkers.

13. The non-transitory computer-readable medium of claim 11, wherein the correlation combines two or more of the simple regression, the ROC curve area maximization, the topology stabilization, and the Spatial Proximity analysis.

14. The non-transitory computer-readable medium of claim 11, wherein the first and second patient samples include at least one of blood samples, urine samples, or tissue samples.

15. The non-transitory computer-readable medium of claim 11, wherein the disease diagnosed is one of prostate cancer, breast cancer, lung cancer, or ovarian cancer.

16. The non-transitory computer-readable medium of claim 15, wherein the disease diagnosed is the stage of the prostate cancer, breast cancer, lung cancer, or ovarian cancer based on Gleason Score.

17. The non-transitory computer-readable medium of claim 16, wherein the first and second patient samples comprise cancer stage data, and wherein the cancer stage data is categorized into a plurality of binary groups.

18. The non-transitory computer-readable medium of claim 7, wherein each of the binary groups is scored.

19. The non-transitory computer-readable medium of claim 11, wherein the biomarkers are selected from a functional group of cytokines, and wherein the functions of the cytokines are at least three of: pro-inflammatory, anti-inflammatory, anti-tumor genesis, cell apoptosis, and vascularization.

20. The non-transitory computer-readable medium of claim 11, wherein the first biomarker is VEGF.
